(19) United States
(12) Patent Application Publication
(10) Pub. No.: US 2025/0320568 A1
(43) Pub. Date: Oct. 16, 2025

(54) MULTIPLEX BIOSENSOR FOR RAPID POINT-OF-CARE DIAGNOSTICS

(71) Applicant: HeMemics Biotechnologies, Inc., Gaithersburg, MD (US)

(72) Inventors: David Ho, McLean, VA (US); Srivatsa Aithal, Clarksburg, MD (US); Rhitvik Kumawat, Germantown, MD (US); Ankit Kumar, Germantown, MD (US); Khanh Duong, Germantown, MD (US); John Warden, Chevy Chase, MD (US)

(21) Appl. No.: 18/866,404

(22) PCT Filed: May 16, 2023
(86) PCT No.: PCT/US2023/067067

§ 371 Date: Nov. 15, 2024

Related U.S. Application Data
(60) Provisional application No. 63/342,248, filed on May 16, 2022.
(60) Provisional application No. 63/486,144, filed on Feb. 21, 2023.

(51) Int. Cl. C12Q 1/70; C12Q 1/6825; G01N 27/02

(52) U.S. Cl. CPC C12Q 1/701; C12Q 1/6825; G01N 27/028; G01N 2333/08; G01N 2469/10

(57) ABSTRACT

The present disclosure relates to carbon-based biosensors and biosensor systems. The disclosure further relates to methods of rapidly detecting a target material in a biological sample using the biosensor and biosensor systems described herein to characterize a pathogen's antigen profile and/or a subject's immune response to pathogen exposure, providing an innovative point-of-care biosensor device.

### CROSS-REFERENCE TO RELATED APPLICATIONS

This application claims the benefit of U.S. Provisional Application No. 63/342,248 filed May 16, 2022 and U.S. Provisional Application No. 63/486,144 filed Feb. 21, 2023, both of which are incorporated by reference in their entireties.

### FIELD

The present disclosure is directed to biosensors, systems, and methods capable of rapid detection of a target material in a biological sample.

### BACKGROUND

The COVID-19 public health emergency highlighted the nation's need for next-generation diagnostics that can be easily deployed in both traditional care environments and the field. Delayed results, inaccurate reporting, and in some cases, inaccessibility to testing stymied the reopening of the economy and encouraged the spread of COVID-19.

Rapid, cost-effective, and real-time biomarker measurements are essential steps toward realizing the goal of quickly and effectively diagnosing emerging illnesses, like COVID-19 and other viruses. Currently, many biological assays rely on labeled detector molecules and optical-based detectors for diagnosis. The cost and time delay associated with these methods radically impacts patient outcomes, as testing, consultation and treatment are typically spread over several interactions. An innovative point-of-care biosensor device that can provide rapid, accurate disease detection is urgently needed.

The present invention is directed to overcoming these and other deficiencies in the art.

### SUMMARY

In some embodiments, a biosensor is provided, comprising an anti-static substrate comprising a planar surface; at least one spatially defined active area on the planar surface of the anti-static substrate, each active area comprising a carbon material, a first and a second signal electrode in operable contact with the carbon material, and at least one gate electrode; a plurality of capture molecules, wherein different capture molecules are positioned at different active areas and immobilized on the deposited carbon material of the active areas; and an electrical connection comprising a plurality of electrical contacts, each electrical contact configured to transmit an electrical signal between the first and second signal electrodes and at least one gate electrode of a single active area, and the electrical connection.

In some embodiments, a biosensor is provided, comprising an anti-static substrate comprising a planar surface; at least one spatially defined active area on the planar surface of the anti-static substrate, each active area comprising a carbon material, a first, second, and a third signal electrode in operable contact with the carbon material, and at least one gate electrode; a plurality of capture molecules, wherein different capture molecules are positioned at different active areas and immobilized on the deposited carbon material of the active areas; and an electrical connection comprising a plurality of electrical contacts, each electrical contact configured to transmit an electrical signal between the first and second signal electrodes and at least one gate electrode of a single active area, and the electrical connection.

In some embodiments, a biosensor is provided, comprising an anti-static substrate comprising a planar surface; at least one spatially defined active area on the planar surface of the anti-static substrate, each active area comprising a carbon material, a first, a second, a third, and a fourth signal electrode in operable contact with the carbon material, and at least a first and a second gate electrode; a plurality of capture molecules, wherein different capture molecules are positioned at different active areas and immobilized on the deposited carbon material of the active areas; and an electrical connection comprising a plurality of electrical contacts, each electrical contact configured to transmit an electrical signal between the first and second signal electrodes and at least one gate electrode of a single active area, and the electrical connection.

In some embodiments, a biosensor system is provided for characterizing a subject's immune response to pathogen exposure, comprising an electronic reader comprising a circuit for delivering a signal; and a processing device for reading the signal; any biosensor disclosed herein operatively connected to the electronic reader via the electrical connection of the biosensor and configured to receive the signal delivered by the circuit. In some embodiments, the electronic reader is configured to deliver the signal to the biosensor and obtain an output impedance value before and after a sample has been applied to the array of active areas on the biosensor, and said processing device is configured to compare the output impedance values to determine whether a binding event has occurred at one or more of the active areas to characterize the subject's immune response to pathogen exposure.

In some embodiments, a method of characterizing a subject's immune response to pathogen exposure is provided, the method comprising: collecting a biological sample from a subject; providing a biosensor system described herein; delivering an electrical signal to the biosensor via the circuit of the electronic reader; determining a base resistance between the first and second signal electrodes at each active area on the biosensor; applying the biological sample from the subject to at least one active area on the biosensor, such that the biological sample is in operable contact with the carbon material between the first and second signal electrodes, and the at least one gate electrode at the at least one active area; identifying a change in the base resistance between the first and second signal electrodes, resulting from applying the biological sample to the at least one active area; and characterizing the subject's immune response to the pathogen, or the pathogen's antigen profile based on the change in the base resistance between the first and second signal electrodes at the at least one active area.

In some embodiments, a method of characterizing a subject's immune response to pathogen exposure, the method comprising: collecting a biological sample from a subject; providing any biosensor system disclosed herein; delivering an electrical signal to the biosensor via the circuit of the electronic reader; applying a control solution to at least one active area on the biosensor, such that the control solution is in operable contact with the carbon material between the first and the third signal electrodes, and the at least one gate electrode at the at least one active area; determining a base resistance between the second and third signal electrodes at the at least one active area; applying the biological sample from the subject to the at least one active area, such that the biological sample is in operable contact with the carbon material between the second and third signal electrodes; identifying a change in the base resistance between the second and third signal electrodes resulting from applying the biological sample to the at least one active area; and characterizing the subject's immune response to the pathogen, or the pathogen's antigen profile based on the change in the base resistance between the second and third signal electrodes at the at least one active area.

In some embodiments, a method of characterizing a subject's immune response to pathogen exposure, the method comprising: collecting a biological sample from a subject; providing any biosensor system disclosed herein; delivering an electrical signal to the biosensor via the circuit of the electronic reader; applying a first control solution to at least one active area on the biosensor, such that the first control solution is in operable contact with the carbon material between the first and the third signal electrodes, and the first gate electrode at the at least one active area; applying a second control solution to the at least one active area, such that the second control solution is in operable contact with the carbon material between the second and the fourth signal electrodes, and the second gate electrode at the at least one active area; determining a base resistance between the third and fourth signal electrodes at the at least one active area; applying the biological sample from the subject to the at least one active area, such that the biological sample is in operable contact with the carbon material between the second and third signal electrodes; identifying a change in the base resistance between the second and third signal electrodes resulting from applying the biological sample to the at least one active area; and characterizing the subject's immune response to the pathogen, or the pathogen's antigen profile based on the change in the base resistance between the second and third signal electrodes at the at least one active area.

In some embodiments, a method of making a biosensor is provided, wherein the method comprises: obtaining a substrate with at least two layers, wherein there is a z-height difference between the at least two layers, transferring graphene onto the substrate, wherein the graphene breaks along the at least two layers due to the z-height difference, washing the biosensor to remove excess graphene, and, optionally, adding additional layers to the biosensor. In some embodiments, the z-height difference between the at least two layers is about 50 nanometers to about 3 millimeters. In some embodiments, the substrate comprises at least 3 layers. In some embodiments, the substrate comprises at least 4 layers. In some embodiments, the additional layers in the method comprise insulating material, or electrode material, or both. In some embodiments, any of the biosensors described herein can be made with this contour ablation method.

The biosensor described herein harnesses the superior electric charge capabilities of graphene to deliver a nearly instantaneous (˜60 seconds), highly sensitive point-of-care testing platform capable of detecting up to a dozen unique antibody/antigen pairs from a single drop of saliva. This allows for testing, diagnosis, and treatment in one interaction, resulting in more accurate, effective treatment plans and vastly improved patient outcomes.

### BRIEF DESCRIPTION OF THE DRAWINGS

FIG. 1 is a schematic cross-sectional view of an active area on an exemplary biosensor device as described herein.

FIG. 2 is another schematic cross-sectional view of an active area on an exemplary biosensor device as described herein.

FIG. 3 is another schematic cross-sectional view of an active area on an exemplary biosensor device as described herein.

FIG. 4 is another schematic cross-sectional view of an active area on an exemplary biosensor device as described herein.

FIG. 5 is another schematic cross-sectional view of an active area on an exemplary biosensor device as described herein.

FIG. 6 is another schematic cross-sectional view of an active area on an exemplary biosensor device as described herein.

FIG. 7 is a top-down view of a section of a biosensor device as described herein showing an array of active areas.

FIG. 8 is a top-down view of a section of a biosensor device as described herein showing multiple arrays of active areas.

FIG. 9 is an electrical circuit diagram illustrating the electrical circuitry associated with an array of active areas on a biosensor device as described herein.

FIG. 10 is a schematic view of an exemplary electronic reader for use in combination with the biosensor described herein.

FIG. 11 is a block diagram of an exemplary circuit that may be used in combination with the electronic reader of FIG. 6.

FIG. 12 is a flowchart of an exemplary process for detecting a target moiety using a biosensor and electronic reader as described herein.

FIGS. 13A-13C show the method of detecting target antigen in a sample using a biosensor containing an electromagnetic substrate as described herein. As shown in the cross-sectional view of the sensor provided in FIG. 13A, the electromagnet is positioned beneath the substrate of the biosensor, and the antibody (or other biological detecting agent) is immobilized to the active areas on the surface of the substrate. A drop of high viscosity fluid containing antigen complexed to magnetic beads is applied to active areas on the surface. In the depiction of FIG. 13A, the electromagnet is turned off. Absorption of magnetic bead-antigen complex onto the surface of active areas on the biosensor when the electromagnet is turned on is depicted in FIG. 13B. When the electromagnet is turned off, unbound magnetic bead-antigen complex is released from the circuit, whereas antigen-bead complex that is specifically bound to the immobilized antibody (or other biological detecting agent) will remain bound to the circuit and be detected (FIG. 13C).

FIG. 14 is a graph of circuit resistance over the 7-step adhesion process. During this process protein or antibody was immobilized on the surface of the graphene sensor.

FIG. 15 is a graph showing the change in Dirac point voltage across two circuits containing electromagnets beneath the surface. The far left box shows the Dirac point voltage at baseline. After 594 seconds, 2.5 nm-ferrous oxide magnetic beads conjugated to BSA were added to the circuit and a corresponding increase in signal was observed between 600-800 seconds (middle box). The transition stabilized at about 800 seconds, and a second addition of 2 nm-ferrous oxide magnetic bead conjugated to BSA was made at 1188 seconds. This addition led to another increase in voltage by about 200 mV over the course of 200 seconds (far right box).

FIGS. 16A-16F are schematic cross-sectional views of an active area on an exemplary biosensor device as described herein, wherein the carbon material overlaid on top is broken into several sections during the assembly of the biosensor.

FIGS. 17A-17F are also schematic cross-sectional views of an active area on an exemplary biosensor device as described herein, wherein the carbon material overlaid on top is broken into several sections during the assembly of the biosensor.

FIG. 18 is a graph showing the detection of ricin from spike mud samples using a biosensor manufactured with a contour ablation process.

FIG. 19 is a graph showing the detection of respiratory syndrome virus (PRRSV) from spike saliva samples. The mismatched RNA is from influenza virus samples.

### DETAILED DESCRIPTION

The present disclosure is directed to biosensors, systems, and methods capable of rapid detection of a target material in a biological sample.

In some embodiments, the biosensor disclosed herein comprises a substrate that comprises anti-static substrate and a spatially defined array of active areas on the planar surface of the substrate. Each active area on the planar surface comprises a carbon material; a first and a second signal electrode in operable contact with the carbon material, and at least one gate electrode. The biosensor further comprises a plurality of different detecting agents positioned at different active areas and immobilized on the deposited carbon material of the active areas; and an electrical connection comprising a plurality of electrical contacts, each electrical contact configured to transmit an electrical signal between the first and second signal electrodes and the at least one gate electrode of a single active area and the electrical connection.

In some embodiments, the biosensor disclosed herein comprises a substrate that comprises anti-static substrate and a spatially defined array of active areas on the planar surface of the substrate. Each active area on the planar surface comprises a carbon material; a first, a second, and a third signal electrode in operable contact with the carbon material, and at least one gate electrode. The biosensor further comprises a plurality of different detecting agents positioned at different active areas and immobilized on the deposited carbon material of the active areas; and an electrical connection comprising a plurality of electrical contacts, each electrical contact configured to transmit an electrical signal between the first, second, and third signal electrodes and the at least one gate electrode of a single active area and the electrical connection.

In some embodiments, the biosensor disclosed herein comprises a substrate that comprises anti-static substrate and a spatially defined array of active areas on the planar surface of the substrate. Each active area on the planar surface comprises a carbon material; a first, a second, a third, and a fourth signal electrode in operable contact with the carbon material, and at least a first and a second gate electrode. The biosensor further comprises a plurality of different detecting agents positioned at different active areas and immobilized on the deposited carbon material of the active areas; and an electrical connection comprising a plurality of electrical contacts, each electrical contact configured to transmit an electrical signal between the first, second, third, and fourth signal electrodes and the at least a first and the at least a second gate electrode of a single active area and the electrical connection.

The basic structure of the biosensor disclosed herein is described in International Patent Application Publication No. WO2020072966 to Hememics Biotechnologies, Inc., which is hereby incorporated by reference in its entirety.

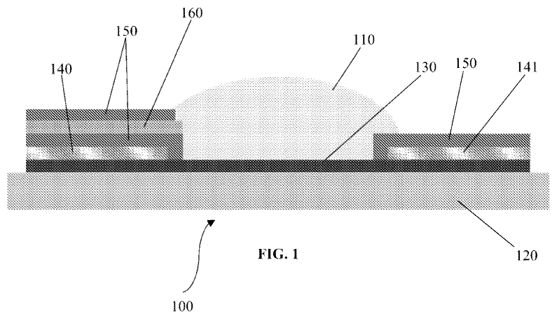

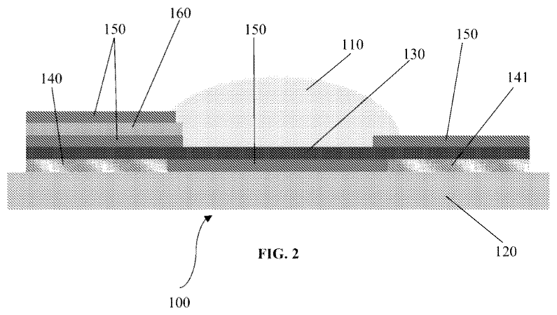

The schematic illustrations of FIGS. 1 and 2 provide cross-sectional views of an active area 100 on a biosensor as described herein, wherein the biosensor has two signal electrodes. In reference to FIGS. 1 and 2, the biosensor comprises a substrate 120 having a planar surface.

In some embodiments, the biosensor comprises a single-layer substrate. In accordance with this embodiment, the single-layer substrate is a polymeric material. Suitable polymeric materials include, without limitation, poly(methyl methacrylate) (PMMA), polycarbonates (PC), epoxy-based resins, copolymers, polysulfones, elastomers, cyclic olefin copolymer (COC), nylon, polypropylene, a polyester film, polyethylene terephthalate (PET), polyvinyl chloride, polytetrafluoroethylene, and polymeric organosilicons. In any embodiment, the polymeric substrate is modified with an anti-static agent to exhibit suitable anti-static properties to dissipate electrical charge. The anti-static agent can be mixed directly with the polymer material or applied to the surface of the polymer material to impart anti-static quality to the material. Anti-static agents that can be added to polymers to minimize static electricity are known in the art and include, without limitation, fatty acid esters, long chain aliphatic amines and amides, ethoxylated amines, quaternary ammonium compounds (e.g., behentrimonium chloride or cocamidopropyl betaine), esters of phosphoric acid, polyethylene glycol esters, alkylsulfonates, and alkylphosphates. A suitable anti-static quality for a substrate of the biosensor as described herein is determined by the surface resistivity of the material, which is measured in ohms/square. Suitable anti-static polymeric substrate materials of the biosensor have a surface resistivity of between 10³-10¹⁴ ohms/square. In some embodiments, the anti-static polymeric substrate material of the biosensor has a surface resistivity of between 10³-10⁵ ohms/square. In some embodiments, the anti-static polymeric substrate material of the biosensor has a surface resistivity of between 10⁷-10¹⁴ ohms/square.

In reference to FIGS. 1 and 2, each active area 100 on a biosensor functions as a field-effect transistor (FET) sensor unit with a liquid gate. Each active area comprises a conductive carbon material 130 (e.g., graphene) deposited on the planar surface of the substrate 120 between a first signal electrode 140 and a second signal electrode 141. Suitable conductive carbon materials of the active areas include, without limitation, graphene, carbon nanotubes, fullerene or a combination thereof. The area between the electrodes can alternatively comprise other conductive materials known in the art, including, without limitation, silicon, molybdenum disulfide, black phosphorous, and/or metal dichalcogenides.

In some embodiments, the carbon material is graphene polycrystal. In some embodiments, the carbon material is graphene monocrystal. In some embodiments, the carbon material is a single layer. In some embodiments, the carbon material has multiple layers. In some embodiments, the carbon material can be laser or mechanically ablated to create distinct sensors areas approximately 50 μm to 500 μm in width and 100 μm to 3000 μm in length. In some embodiments, the carbon material can be applied to the biosensor by screen printing, rotogravure printing, photolithography, mechanical ablation, or contour ablation. In some embodiments, the carbon material can be applied to the biosensor by screen printing directly onto the anti-static substrate. In some embodiments, the carbon material can be applied to the biosensor by rotogravure printing directly onto the anti-static substrate.

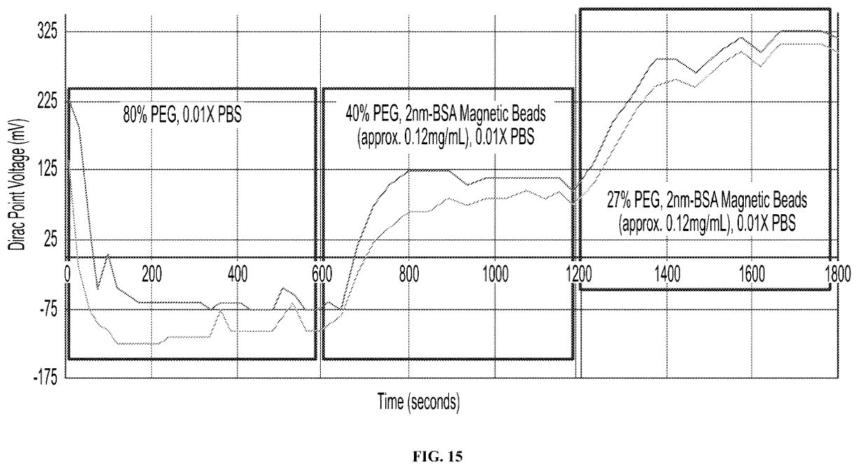

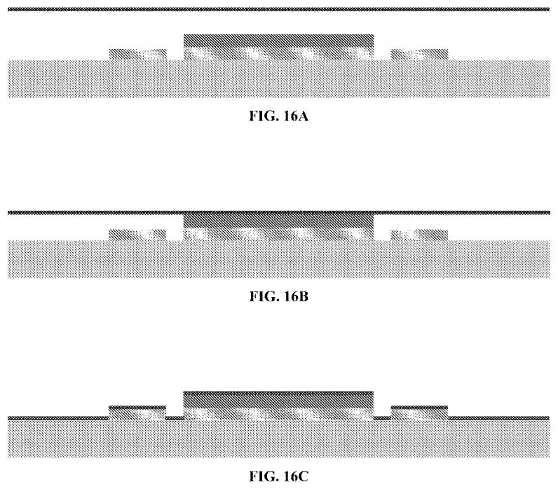

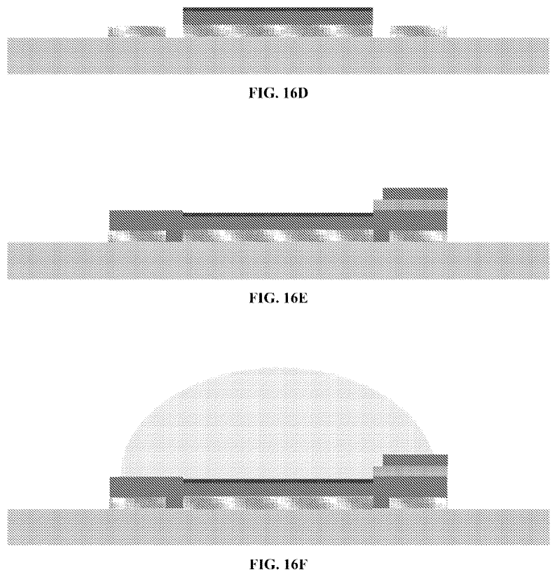

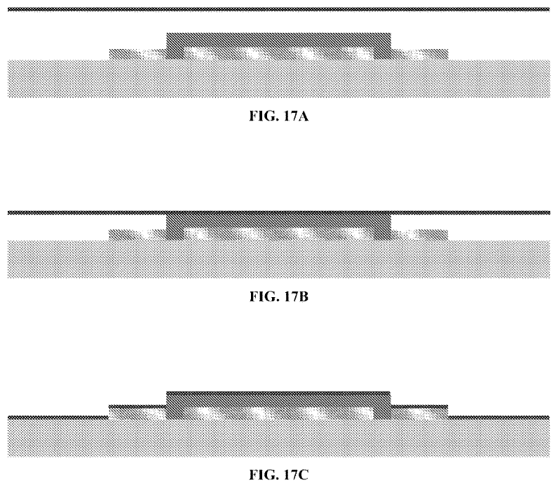

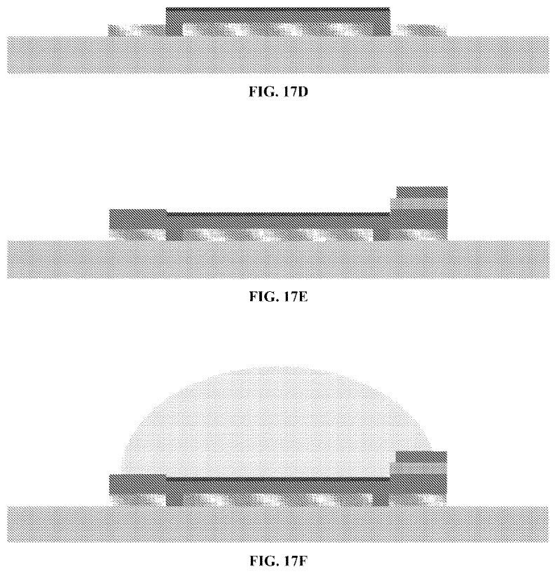

In some embodiments, the carbon material can be transferred to the substrate such that the carbon material is not a continuous flat surface, but a series of surfaces. For example, a carbon material can be transferred to the biosensor via a contour ablation process, as depicted in FIGS. 16A-16F and 17A-17F. As the carbon material is transferred, the height differences of the biosensor break the carbon material, allowing the carbon material to cover the any exposed area of the biosensor at the time of its application.

In some embodiments, height differences can range from about 50 nanometers to about 3 millimeters. In some embodiments, height differences can range from about 100 nanometers to about 3 millimeters. In some embodiments, height differences can range from about 150 nanometers to about 3 millimeters. In some embodiments, height differences can range from about 200 nanometers to about 3 millimeters. In some embodiments, height differences can range from about 250 nanometers to about 3 millimeters. In some embodiments, height differences can range from about 300 nanometers to about 3 millimeters. In some embodiments, height differences can range from about 400 nanometers to about 3 millimeters. In some embodiments, height differences can range from about 500 nanometers to about 3 millimeters. In some embodiments, height differences can range from about 600 nanometers to about 3 millimeters. In some embodiments, height differences can range from about 700 nanometers to about 3 millimeters. In some embodiments, height differences can range from about 800 nanometers to about 3 millimeters. In some embodiments, height differences can range from about 900 nanometers to about 3 millimeters. In some embodiments, height differences can range from about 1 millimeter to about 3 millimeters. In some embodiments, height differences can range from about 1.5 millimeters to about 3 millimeters. In some embodiments, height differences can range from about 2 millimeters to about 3 millimeters. In some embodiments, height differences can range from about 2.5 millimeters to about 3 millimeters. In some embodiments, height differences can range from about 50 nanometers to about 2.5 millimeters. In some embodiments, height differences can range from about 50 nanometers to about 2 millimeters. In some embodiments, height differences can range from about 50 nanometers to about 1.5 millimeters. In some embodiments, height differences can range from about 50 nanometers to about 1 millimeter. In some embodiments, height differences can range from about 50 nanometers to about 900 nanometers. In some embodiments, height differences can range from about 50 nanometers to about 800 nanometers. In some embodiments, height differences can range from about 50 nanometers to about 700 nanometers. In some embodiments, height differences can range from about 50 nanometers to about 600 nanometers. In some embodiments, height differences can range from about 50 nanometers to about 500 nanometers. In some embodiments, height differences can range from about 50 nanometers to about 400 nanometers. In some embodiments, height differences can range from about 50 nanometers to about 300 nanometers. In some embodiments, height differences can range from about 50 nanometers to about 250 nanometers. In some embodiments, height differences can range from about 50 nanometers to about 200 nanometers. In some embodiments, height differences can range from about 50 nanometers to about 150 nanometers. In some embodiments, height differences can range from about 50 nanometers to about 100 nanometers.

In some embodiments, the contour ablation process proceeds in several steps, shown in FIGS. 16A-16F and 17A-17F. First, a substrate such as PCB is manufactured various layers, which can include signal electrodes and/or insulating materials, wherein the z-height difference of these layers could range from about 50 nanometers to about 3 millimeters (FIGS. 16A and 17A). In the second step, the graphene grown on a donor substrate is transferred onto this PCB substrate (FIGS. 16B and 17B). Then, the graphene pattern with defined islands of graphene is shown as the graphene breaks due to the z-height difference on the substrate (FIGS. 16C and 17C). Excess graphene can be washed away, leaving only graphene bound to certain areas of the biosensors, for example, on insulating material laid down on the PCB substate (FIGS. 16D and 17D). Finally, additional insulating materials and electrodes can be printed on top, completing the biosensor (FIGS. 16E and 17E, without a sample, and FIGS. 16F and 17F, with a sample on the biosensor). In some embodiments, the contour ablation process can be used to manufacture any of the biosensors described herein.

In some embodiments, a method of making a biosensor is provided, wherein the method comprises: obtaining a substrate with at least two layers, wherein there is a z-height difference between the at least two layers, transferring graphene onto the substrate, wherein the graphene breaks along the at least two layers due to the z-height difference, washing the biosensor to remove excess graphene, and, optionally, adding additional layers to the biosensor. In some embodiments, the z-height difference between the at least two layers is about 50 nanometers to about 3 millimeters. In some embodiments, the substrate comprises at least 3 layers. In some embodiments, the substrate comprises at least 4 layers. In some embodiments, the additional layers in the method comprise insulating material, or electrode material, or both.

In some embodiments, the first signal electrode 140 serves as a source electrode, and second signal electrode 141 serves as a drain electrode, or vice versa. The electrodes each comprise a conductive metal, such as, for example and without limitation gold (Au), copper (Cu), silver (Ag), cobalt (Co), platinum (Pt), titanium (Ti), platinum (Pt), Iridium (Ir), any oxides thereof, and combinations thereof. As show in FIGS. 1 and 2, the electrodes can be encapsulated in an insulating material 150. In some embodiments, the insulating material can be applied to the biosensor by screen printing, rotogravure printing, or photolithography. Each active area of a biosensor further comprises a gate conductor (not shown), which controls the liquid gate, i.e., the electrical field in the ionic fluid sample 110 that is applied to the graphene surface 130 during use of the sensor.

A collection of detecting agents are immobilized to the surface of the carbon material 130, preferably in the presence of a preservative solution as disclosed herein. Suitable detecting agents, preservative agents, and methods of immobilizing the detecting agents to the surface of the carbon material are described herein.

In reference to FIGS. 1 and 2, each active area 100 comprises at least a first gate electrode 160. In some embodiments, the gate electrode 160 is located approximately on top of either the first or second signal electrodes 140, 141. In some embodiments, the gate electrode 160 is mostly covered by an insulating material 150. At least a portion of the gate electrode 160 is designed to contact the ionic fluid sample 110. Without wishing to be bound by theory, the faradic gate current passing through the sample in a conventional FET biosensor remains an important source of error and can damage the detecting agents present on graphene. In some embodiments, the one or more gate electrodes on the biosensor are capable of directly measuring the ionic conductance of the ionic fluid sample 110. This measurement can be used by the gate conductor to apply a gate voltage to the carbon material. In some embodiments, the gate electrodes can apply a gate voltage. In some embodiments, the gate electrode can reduce the level of electronic noise in the system by a factor of 1, 2, 3, 4, 5, 6, 7, 8, 9, or 10. In some embodiments, the gate electrode 160 is capable of applying a gate voltage.

As shown in FIG. 1, the first and second signal electrodes 140, 141 are located on top of the carbon material 130. As shown in FIG. 2, the first and second signal electrodes 140, 141 are located between the anti-static substrate 120 and the carbon material 130. In some embodiments, insulating material 150 may be placed between the first and second signal electrodes 140, 141 so that the carbon material 130 is resting on an even surface.

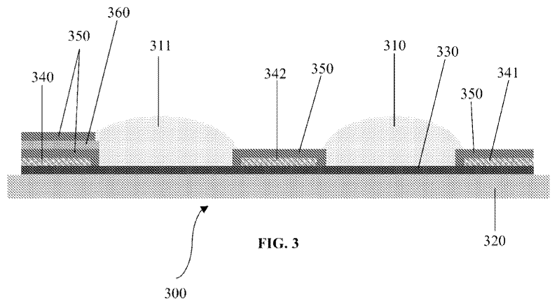

The schematic illustrations of FIGS. 3 and 4 provide cross-sectional views of an active area 300 on a biosensor as described herein, wherein the biosensor has three signal electrodes. In reference to FIGS. 3 and 4, the biosensor comprises a substrate 320 having a planar surface. In some embodiments, the substrate is any substrate described herein.

In reference to FIGS. 3 and 4, each active area 300 on a biosensor functions as a field-effect transistor (FET) sensor unit with a liquid gate. Each active area comprises a conductive carbon material 330 (e.g., graphene) deposited on the planar surface of the substrate 320. A first signal electrode 340 and a second signal electrode 341 are located on either end of the carbon material 330, with a third signal electrode 342 located on top of the carbon material 330 between the first signal electrode 340 and the second signal electrode 341. The carbon material 330 is any conductive carbon material disclosed herein. The area between the electrodes can alternatively comprise other conductive materials known in the art, including, without limitation, silicone, molybdenum disulfide, black phosphorous, and/or metal dichalcogenides.

In some embodiments, the first signal electrode 340 serves as a source electrode, and the second and/or third signal electrode 341, 342 serves as a drain electrode. In some embodiments, the third signal electrode 342 serves as a source electrode, and the first and/or second signal electrode 340, 341 serves as a drain electrode. In some embodiments, the electrodes each comprise any conductive material described herein. As show in FIGS. 3 and 4, any of the electrodes can be encapsulated in an insulating material 350. Each active area of a biosensor further comprises a gate conductor (not shown), which controls the liquid gate, i.e., the electrical field in the ionic fluid sample 310 and the control solution 311 that are applied to the graphene surface 330 during use of the sensor.

A collection of detecting agents are immobilized to the surface of the carbon material 330, preferably in the presence of a preservative solution as disclosed herein. Suitable detecting agents, preservative agents, and methods of immobilizing the detecting agents to the surface of the carbon material are described herein.

In reference to FIGS. 3 and 4, each active area 300 comprises at least a first gate electrode 360. In some embodiments, the gate electrode 360 is located approximately on top of either the first or second signal electrodes 340, 341. In some embodiments, the gate electrode 360 is mostly covered by an insulating material 350. At least a portion of the gate electrode 360 is designed to contact a control solution 311 that is placed between the first and third signal electrodes 340, 342. Without wishing to be bound my theory, the gate electrode 360 is capable of directly measuring the ionic conductance of the control solution 311. This measurement can be used by the biosensor system to remove, reduce, or gate the signal noise present in the system when the biological sample 310 is placed between second and third signal electrodes 341, 342. In some embodiments, the gate electrode can reduce the level of electronic noise in the system by a factor of 1, 2, 3, 4, 5, 6, 7, 8, 9, or 10.

As shown in FIG. 3, the first, second, and third signal electrodes 340, 341, 342 are located on top of the carbon material 330. As shown in FIG. 4, the first, second, and third signal electrodes 340, 341, 342 are located between the anti-static substrate 320 and the carbon material 330. In some embodiments, insulating material 350 may be placed between the first, second, and third signal electrodes 340, 341, 342 so that the carbon material 330 is resting on an even surface.

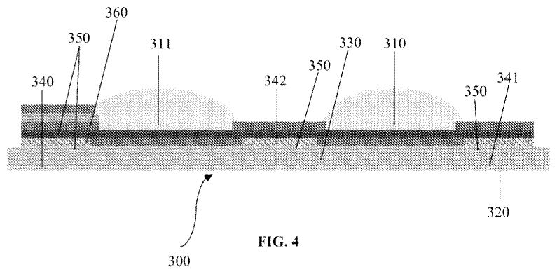

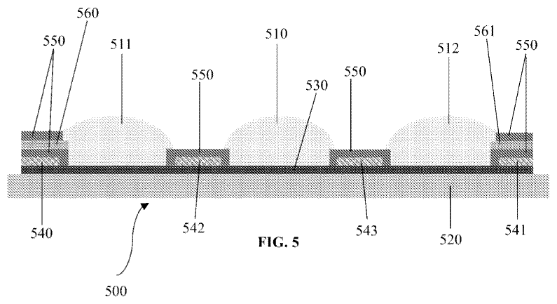

The schematic illustrations of FIGS. 5 and 6 provide cross-sectional views of an active area 500 on a biosensor as described herein, wherein the biosensor has four signal electrodes. In reference to FIGS. 5 and 6, the biosensor comprises a substrate 520 having a planar surface. In some embodiments, the substrate is any substrate described herein.

In reference to FIGS. 5 and 6, each active area 500 on a biosensor functions as a field-effect transistor (FET) sensor unit with a liquid gate. Each active area comprises a conductive carbon material 530 (e.g., graphene) deposited on the planar surface of the substrate 520. A first signal electrode 540 and a second signal electrode 541 are located on either end of the carbon material 530, with a third signal electrode 542 located on top of the carbon material 530 between the first signal electrode 540 and a fourth signal electrode 543, and the fourth signal electrode 543 located on top of the carbon material 530 between third signal electrode 542 and the second signal electrode 541. The carbon material 530 is any conductive carbon material disclosed herein. The area between the electrodes can alternatively comprise other conductive materials known in the art, including, without limitation, silicon, molybdenum disulfide, black phosphorous, and/or metal dichalcogenides.

In some embodiments, at least one of the first, second, third, and fourth signal electrodes 540, 541, 542, 543 serve as a source electrode, while at least one of the remaining signal electrodes serves as a drain electrode. In some embodiments, the electrodes each comprise any conductive material described herein. As show in FIGS. 5 and 6, any of the electrodes can be encapsulated in an insulating material 550. Each active area of a biosensor further comprises a gate conductor (not shown), which controls the liquid gate, i.e., the electrical field in the ionic fluid sample 510, the first control solution 511, and the second control solution 512 that are applied to the graphene surface 530 during use of the sensor.

A collection of detecting agents are immobilized to the surface of the carbon material 530, preferably in the presence of a preservative solution as disclosed herein. Suitable detecting agents, preservative agents, and methods of immobilizing the detecting agents to the surface of the carbon material are described herein.

In reference to FIGS. 5 and 6, each active area 500 comprises at least a first gate electrode 560. In some embodiments, the gate electrode 560 is located approximately on top of either the first or second signal electrodes 540, 541. In some embodiments, the gate electrode 560 is mostly covered by an insulating material 550. At least a portion of the gate electrode 560 is designed to contact the first or second control solution 511, 512 that are placed between the first and third signal electrodes 540, 542, and the second and fourth signal electrodes 541, 543, respectively. Without wishing to be bound by theory, the gate electrode 560 is capable of directly measuring the ionic conductance of either control solution 511, 512. This measurement can be used by the biosensor system to remove, reduce, or gate the signal noise present in the system when the biological sample 510 is placed between third and fourth signal electrodes 542, 543. In some embodiments, the gate electrode can reduce the level of electronic noise in the system by a factor of 1, 2, 3, 4, 5, 6, 7, 8, 9, or 10.

As shown in FIG. 5, the first, second, third, and fourth signal electrodes 540, 541, 542, 543 are located on top of the carbon material 530. As shown in FIG. 6, the first, second, third, and fourth signal electrodes 540, 541, 542, 543 are located between the anti-static substrate 520 and the carbon material 530. In some embodiments, insulating material 550 may be placed between the first, second, third, and fourth signal electrodes 540, 541, 542, 543 so that the carbon material 530 is resting on an even surface.

A top-down view of a section of the biosensor device is provided in FIG. 7. This illustration shows a series of five active areas 700, each active area 700 comprising the conductive carbon material 706 deposited on the substrate. The conductive carbon material 706 forms a channel between the source 708 and drain 710 electrodes of the active area, and is in close proximity to the gate conductor 714. Exemplary dimensions, i.e., length and width, of the carbon material channel of the active area may range from about 10 microns to about 3 mm in length and from about 10 microns to about 1 mm in width. For example, the channel length may range from about 10 microns to about 3 mm, from about 25 microns to 1 mm, from about 50 microns to about 750 microns, from about 75 microns to about 250 microns. In some embodiments, the channel length is about 75 microns, about 80 microns, about 90 microns, or about 100 microns. In some embodiments, the channel length is about 90 microns. Similarly, the width of the channel from side to side may range from about 10 microns to about 1 mm, from about 25 microns to 750 microns, from about 50 microns to about 250 microns, from about 75 microns to about 100 microns. In some embodiments, the channel width is about 75 microns, about 80 microns, about 90 microns, or about 100 microns. In some embodiments, the channel width is about 90 microns. As shown in this embodiment, the gate conductor 714 controls the liquid gate, i.e., the electrical field in the ionic fluid sample applied to each conductive carbon material surface 706 on the device, for all five active areas 700, although other numbers of active areas may be employed. Although shown here are active areas 700 with two signal electrodes, such a set up can be used with active areas with three, four, or more signal electrodes. In some embodiments, multiple liquid gates are present to achieve different patterns of electromagnetic fields across the biosensor.

The biosensor further comprises an electrical connection for operatively connecting the biosensor to an electronic reader. The electrical connection comprises a plurality of electrical contacts where each contact is capable of transmitting an electrical signal between the electrodes of each active area and the electrical connection. In reference to FIG. 7, the electrical contacts include, the shared bonding pads 720 and shared source pad 728. For example, the electrical connection provides current (i.e., electrical signal) from the reader, as described below, to each of a plurality of active areas on the biosensor via the shared source pad 728 and shared source line 724 (i.e., conductive wire). After passing through the source 708 and drain 710 electrodes of an active area, the current is transmitted to the electrical connection via the individual drain lines 722 and drain bonding pads 720. The drain bonding pads 720 of the electrical connection form a circuit with components of the reader device, as described below.

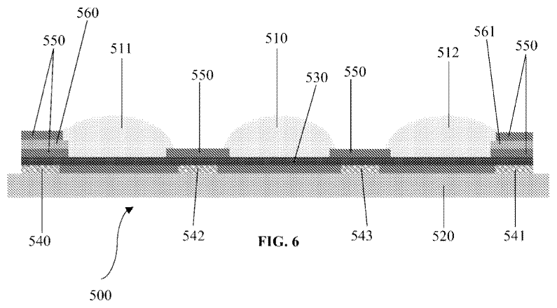

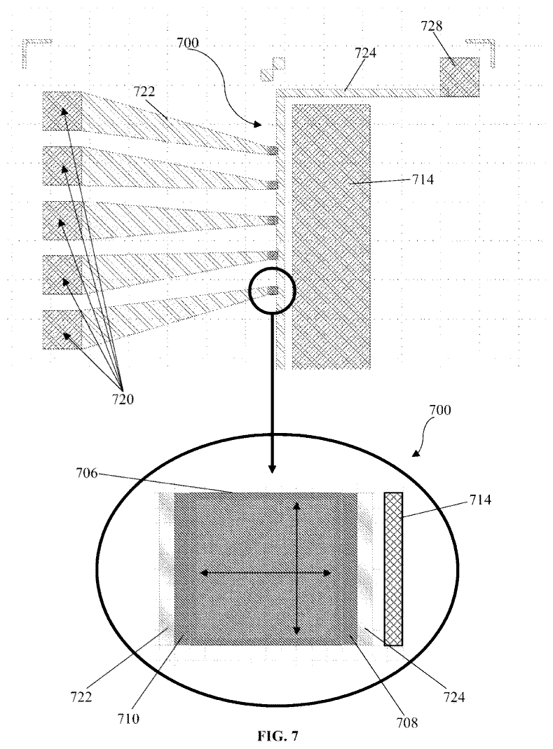

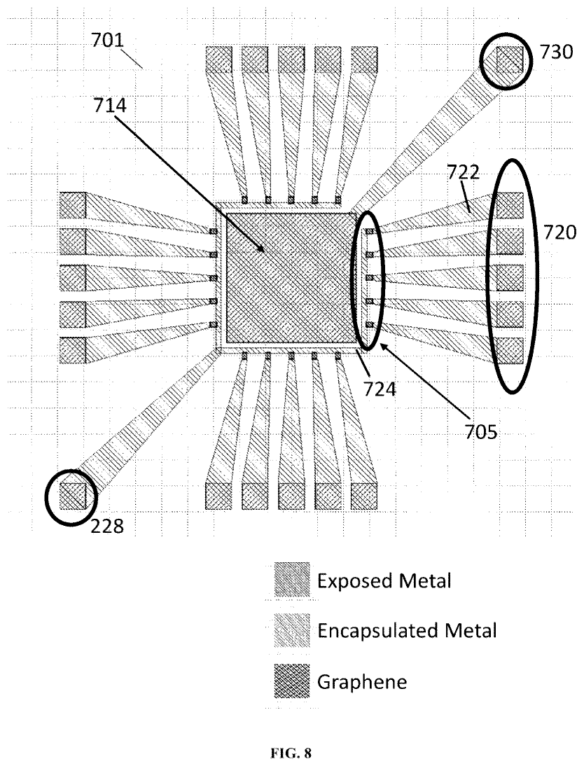

A top-view of a sensing unit 701 of the biosensor is provided in FIG. 8. The sensing unit comprises all of the active areas 700 on a biosensor. In this illustration, the sensing unit 701 of the biosensor comprises four arrays 705 of active areas 700, each array 705 comprising five active areas 700 (as shown in FIG. 7), although other numbers of active areas may be employed. The arrays of active areas are arranged around the periphery of the gate conductor 714. In some embodiments, the gate conductor is at least one order of magnitude greater in size than the dimensions of the carbon material of the active area on the sensor. In some embodiments, the gate conductor is at least two orders of magnitude greater in size than the dimensions of the carbon material of the active area. In some embodiments, the gate conductor is at least three orders of magnitude greater in size than the dimensions of the carbon material of the active area. The larger dimensions of the gate conductor relative to the carbon material of the active area increases the sensitivity of the sensor to detecting voltage changes. Current is fed to each of the twenty active areas 700 via the electrical connection through the shared source pad 728 and shared source line 724. Current is provided to the gate conductor 714 via the electrical connection through the conductor gate bonding pad 730. Any change in current flow through the active area, e.g., an increase in resistance resulting from the presence and binding of a target moiety in a sample applied to the sensor to the immobilized biological detecting agent on the surface of the graphene, is transmitted to the electrical connection of the sensor by the individual drain lines 722 and drain bonding pads 720.

FIG. 9 shows an electrical circuit diagram illustrating aspects of FIG. 8. In this diagram, the graphene active areas 906 are modeled as resistors, receiving current from the shared source 924. In this illustration, the individual drain lines and drain pads connect to a multiplexer 936. The multiplexer 936 acts as a selector, selectively retrieving signal and transmitting the signal to the electrical connection for further transmission to a detector 938 in the reader.

As shown in FIG. 8, the sensing unit of a biosensor as disclosed herein comprises a plurality of active areas on the planar surface of the substrate to facilitate multiplex detection of different target moieties. As will be understood by one of skill in the art, a biosensor as described herein may comprise at least 10, at least 20, at least 30, at least 40, at least 50, at least 60, at least 70, at least 80, at least 90, at least 100, at least 125, at least 150, at least 175, at least 200, at least 225, at least 250, at least 275, at least 300, at least 325, at least 350, at least 375, at least 400, at least 425, at least 450, at least 475, at least 500 or more active areas.

Deposited on the carbon or other conductive carbon material of each active unit is a collection of detecting agents. In one embodiment, the active units across the biosensor device each contain a collection of different detecting agents, where the detecting agents are pathogen proteins or peptides thereof, or polynucleotides such as DNA, RNA, oligonucleotides and modified nucleotide sequences. In accordance with this embodiment, each of the different detecting agents are positioned at different active areas and immobilized on the deposited carbon material of said active areas across the biosensor surface. In some embodiments, the plurality of detecting agents are derived from one or more infectious agents selected from a virus, a bacterium, or a combination thereof. Suitable pathogen proteins or peptide thereof for immobilizing on the graphene surface are generally between 5 and 100 amino acid residues in length, 5 and 75 amino acid residues in length, 5 and 50 amino acid residues in length, 10 and 50 amino acid residues in length, 15 and 50 amino acid residues in length, 20 and 50 amino acid residues in length, 25 and 50 amino acid residues in length, 30 and 50 amino acid residues in length, 35 and 50 amino acid residues in length, 40 and 50 amino acid residues in length, 45 and 50 amino acid residues in length, 45 and 75 amino acid residues in length, or 45 and 100 amino acid residues in length. Suitable polynucleotides for immobilizing on the graphene surface are generally between 5 and 100 nucleic acid residues in length, 5 and 75 nucleic acid residues in length, 5 and 50 nucleic acid residues in length, 10 and 50 nucleic acid residues in length, 15 and 50 nucleic acid residues in length, 20 and 50 nucleic acid residues in length, 25 and 50 nucleic acid residues in length, 30 and 50 nucleic acid residues in length, 35 and 50 nucleic acid residues in length, 40 and 50 nucleic acid residues in length, 45 and 50 nucleic acid residues in length, 45 and 75 nucleic acid residues in length, or 45 and 100 nucleic acid residues in length.

In some embodiments, the plurality of detecting agents are derived from one or more viruses, including, but not limited to SARS-COV-2, Influenza A, Influenza B, Human papilloma virus, Venezuelan equine encephalitis virus, Vaccinia virus, Ebola virus, Lassa fever virus, Rift Valley fever virus and combinations thereof. The pathogen proteins or peptides can also be derived from parainfluenza, paramyxovirus, adenovirus, parvovirus, enterovirus, variola virus, rotavirus, hemorrhagic fever viruses (viruses in the families of Arenaviridae, Bunyaviridae, Filoviridae, Falviviridae, and Togaviridae) hepatitis virus, parechovirus, human T-lymphotrophic virus, and Epstein-Barr virus (herpes virus).

In one embodiment, the plurality of detecting agents are derived from coronavirus. These include both human coronaviridae virus (e.g., SARS-COV-2, SARS-COV, MERs-COV, HCoV-NL63, HCoV-229E, HCoV-OC43, and HCoV-HKU1) and animal coronaviridae viruses (e.g., Feline CoV [serotypes I and II], porcine epidemic diarrhea CoV (PEDV), porcine PRCV, porcine TGEV, Dog CCOC, Rabbit RaCoV, etc.).

In some embodiments, the plurality of detecting agents are derived from different viruses so as to allow for the multiplex detection of different corresponding antibodies in a biological sample being tested. In some embodiments, the plurality of pathogen proteins or peptides are derived from the same virus, e.g., SARS-COV-2, to comprehensively characterize a subject's immune (i.e., antibody) response to infection by the virus. In some embodiments, the plurality of pathogen proteins or peptides are derived from SARS-COV-2. In some embodiments, the plurality of pathogen proteins or peptides are derived from SARS-COV-2 and Influenza A.

In some embodiments, the plurality of detecting agents are derived from one or more bacteria, including, but not limited to Pseudomonas aeruginosa, Neisseria gonorrhoeae, Chlamydia trachomatis, Treponema pallidum, Bacillus anthracis, Yersinia pestis, Francisella tularensis, Burkholderia pseudomallei, Burkholderia mallei, Enterococcus faecium, Stapyylococcus aureus, Klebsiella pneumonia, Acinetobacter baumannii, Enterobacter species and combinations thereof.

In one embodiment, the collection of detecting agents immobilized on the deposited material of said active area comprises a collection of binding molecules. Suitable binding molecules for immobilization on the active areas of the biosensor encompass any biological material that serves as a binding partner or pair to a detectable target material present or potentially present in a biological sample. In some embodiments, the binding molecules of the collection are antibody-based molecules. An antibody-based molecule as used herein includes, without limitation full antibodies, epitope binding fragments of whole antibodies, and antibody derivatives.

Full antibodies include intact immunoglobulins comprising two heavy chains and two light chains, each of these chains comprising a variable region (i.e., V_{H} and V_{L}) and constant region (i.e., C_{H} and C_{L}). Epitope binding fragments of antibodies (including Fab and (Fab)₂ fragments) that exhibit epitope-binding that are suitable for immobilization on the active area of the biosensor include without limitation (i) Fab′ or Fab fragments, which are monovalent fragments containing the V_{L}, V_{H}, C_{L} and C_{H}1 domains; (ii) F(ab′)₂ fragments, which are bivalent fragments comprising two Fab fragments linked by a disulfide bridge at the hinge region; (iii) Fd fragments consisting essentially of the V_{H} and CHI domains; (iv) Fv fragments consisting essentially of a V_{L} and V_{H} domain, (v) dAb fragments, which consist essentially of a V_{H} or V_{L} domain and also called domain antibodies, and (vii) isolated complementarity determining regions (CDR). An epitope-binding fragment may contain 1, 2, 3, 4, 5 or all 6 of the CDR domains of such antibody. Antibody derivatives suitable for immobilization on the active areas of the biosensor include those molecules that contain at least one epitope-binding domain of an antibody and are typically formed using recombinant techniques. One exemplary antibody derivative includes a single chain Fv (scFv). A scFv is formed from the two domains of the Fv fragment, the V_{L} region and the V_{H} region.

In some embodiments, the binding molecules of the collection are antibody mimetics. Exemplary antibody mimetics for immobilization on the biosensor active areas are readily known in the art and include, without limitation, affibodies, affilins, affimers, monobodies, and DARPINs.

Other binding materials suitable for immobilization on the carbon material of the active areas of the biosensors described herein includes, without limitation, carbohydrates, lipids, nucleic acids (DNA, RNA), aptamers, recombinant proteins, hybrid molecules such as protein conjugated to DNA or RNA, DNA conjugated to carbohydrates, molecularly imprinted polymers, etc. Biological binding materials also encompass, for example, whole cells or cell fragments of mammalian cells, prokaryotic cells, parasites, viruses, nucleated or enucleated cells.

The collection of binding molecules immobilized on the carbon surface of the active areas of the biosensor bind one or more pathogenic proteins, including pathogenic proteins from infections agents such as viruses, bacteria, toxins, and combinations thereof. Detection of the pathogenic proteins in a sample via binding to the binding molecules is indicative of the presence of the pathogen in the sample. In some embodiments, the binding molecules of the collection on the biosensor bind to one or more pathogenic proteins from a single infectious agent. In some embodiments, the collection of binding molecules on the biosensor bind to pathogenic proteins from different infectious agents to enable multiplex detection of various pathogens in a single sample (e.g., multiplex detection of viruses, bacteria, and/or toxins).

In some embodiments, the binding molecules of the collection bind one or more pathogenic proteins of a virus. Exemplary viruses include, without limitation, SARS-CoV-2, influenza A, influenza B, human papilloma virus, Venezuelan equine encephalitis virus, Vaccinia virus, Ebola virus, Lassa fever virus, Rift Valley fever virus and combinations thereof. In some embodiments, binding molecules of the collection bind one or more pathogenic proteins of SARS-COV-2. In some embodiments, binding molecules of the collection bind one or more pathogenic proteins of SARS-COV-2 and Influenza A.

In some embodiments, binding molecules of the collection bind one or more pathogenic proteins of bacteria. Exemplary bacteria include, without limitation, Pseudomonas aeruginosa, Neisseria gonorrhoeae, Chlamydia trachomatis, Treponema pallidum, Bacillus anthracis, Yersinia pestis, Francisella tularensis, Burkholderia pseudomallei, Burkholderia mallei, and combinations thereof.

In some embodiments, binding molecules of the collection bind one or more toxins to facilitate detection of the presence of a toxin in a biological sample. Exemplary toxins that can be detected using suitable antibodies include, without limitation, Ricin toxin, Botulinum Toxin A/B/E, Staphylococcus enterotoxin B (SEB), Abrin toxin, T-2 toxin, B. anthracis LF toxin, B. anthracis EF toxin, B. anthracis PA toxin, and combinations thereof.

In some embodiments, the collection of detecting agents immobilized on the deposited material of said active area (e.g., the carbon material) comprises a collection of binding molecules together with a plurality of pathogenic proteins, where different binding molecules and pathogenic proteins are spatially arranged at different active areas on the biosensor surface. The combination of binding molecules (suitable for detecting the presence of pathogenic proteins in a sample) and pathogenic proteins or peptides (suitable for detecting the presence of antibodies in a sample) enables a comprehensive characterization of a biological sample. For example, when the sample is a biological sample from a subject (e.g., a human mucosal, blood, or plasma sample), detecting the presence of pathogenic proteins in the sample indicates the presence of an active infection while detecting the presence of antibodies in the sample indicates previous infection and/or provides information on the immune response mounted against that infection.

In some embodiments, the binding molecules are lyophilized before or after being immobilized on the carbon material. In some embodiments, the binding molecules are lyophilized by the use of a lyophilizing agent such as Hemsol®. In some embodiments, the lyophilization process involves freezing and drying or sublimation process.

In some embodiments, the active areas containing the detecting agents, i.e., the pathogenic proteins or peptide thereof or binding molecules, are contacted with a preservative solution to preserve the integrity and stability of the detecting agents immobilized thereto. In some embodiments, the preservative solution comprises at least one large MW sugar (>40,000 Da) and at least another smaller MW sugar (<40,000 Da). Once added to the active areas containing the detecting agents, the detecting agents are dried to a final moisture content of from about 5% to about 95%. At least one large MW sugar and the at least another small MW sugar can be present in a single preservative solution or may be separate solutions.

In some embodiments, the preservative solution included in the deposited material comprises at least one membrane penetrable sugar, at least one membrane impenetrable sugar, at least one anti-microbial agent, at least one anti-oxidant, optionally a salt, adenosine, and, optionally, albumin. In some embodiments, the preservative solution comprises at least one membrane penetrable sugar (e.g., trehalose and glucose), at least one membrane impenetrable sugar (e.g., dextran, such as dextran-70), at least one anti-microbial agent (e.g., sulfanilamide), at least one anti-oxidant (e.g., mannitol and vitamin E), optionally adenosine, and, optionally, albumin. In some embodiments, the preservative solution comprises at least one membrane penetrable sugar (e.g., trehalose and glucose), at least one membrane impenetrable sugar (e.g., dextran, such as dextran-70), at least one anti-microbial agent (e.g., sulfanilamide), at least one anti-oxidant (e.g., mannitol and vitamin E), adenosine, albumin, a salt (e.g., chloride salts such as KCl, NaCl, CaCl2, and covalent chlorides of metals or nonmetals such as titanium (IV) chloride or carbon tetrachloride), a buffer (e.g., K2HP04), and a chelating agent (e.g., EDTA). Suitable preserving liquids and methods are described in U.S. Pat. Nos. 8,628,960, 9,642,353, and 9,943,075, each of which is incorporated herein in its entirety, or available from HeMemics Biotechnologies, Inc. (Hem Sol™).

In accordance with present disclosure, each of the plurality of detecting agents, i.e., pathogen proteins or peptides and/or binding molecules are immobilized on the deposited carbon material. In some embodiments, the immobilization is via a covalent bonding interaction. In some embodiments, the binding molecule and/or proteins or peptides are attached via a hydrophobic linker, wherein the hydrophobic linker is coupled to the detecting agent's amino or carboxy terminus. In some embodiments, the hydrophobic linker is a peptide linker comprising two or more linker amino acids and one or more aromatic amino acid residues. In some embodiments, the two or more linker amino acid residues are selected from glycine, alanine, serine, and combinations thereof. In some embodiments, the hydrophobic linker comprises a polycyclic aromatic hydrocarbon. A suitable polycyclic aromatic hydrocarbon linker comprises, without limitation, pyrene.

Other methods of immobilizing the detecting agents to the carbon material of the active areas are known in the art and suitable for use in accordance with the biosensor described herein. These include, for example, and without limitation, attachment via 1-ethyl-3-(3-dimethylamino propyl carbodiimide hydrochloride (EDC) and N-hydroxysuccinimide (NHS) (EDC/NHS) chemical reactions, attachment via electrostatic bonding, or via 1-pyrenebutanoic acid succinimidyl ester (PASE) linker (see e.g., Pena-Bahamonde et al., “Recent Advances in Graphene-based Biosensor Technology with Applications in Life Science,” J. Nanobiotechnology 16:75 (2018), which is hereby incorporated by reference in its entirety).

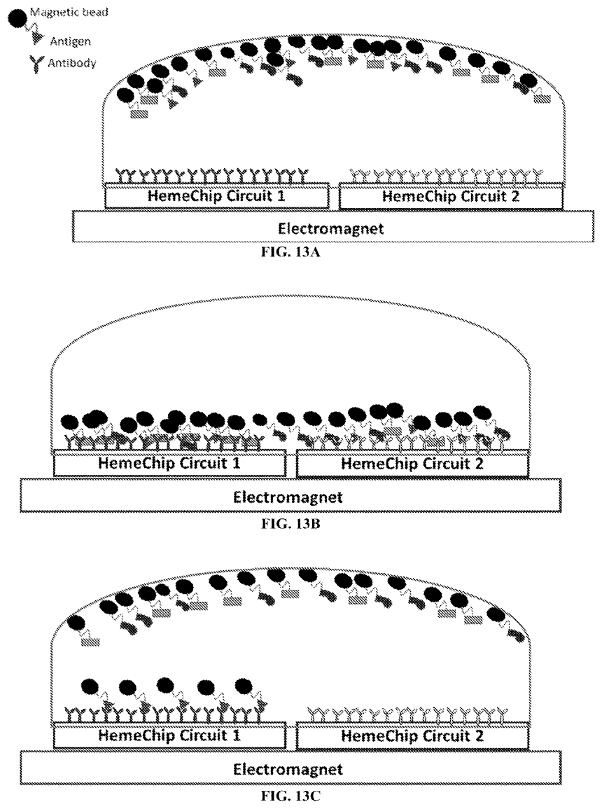

In one aspect, the biosensor of the present disclosure further comprises an electromagnet that is positioned beneath the substrate of the biosensor. The biosensor further comprises a means for turning the electromagnet on and off. A schematic of a biosensor comprising an electromagnet and the sequential steps of sample antigen or antibody detection using the electromagnet is provided in FIGS. 13A-13C.

As described herein, the electromagnet feature of the biosensor used in combination with a sample, where the antigens or antibodies of the sample have been conjugated to magnetic beads, allows user control over the rate of sample antigen/antibody diffusion to the surface of the biosensor, ensuring quick absorption of the antigen/antibody to the active areas of the biosensor surface. This ensures antigens/antibodies in the sample are brought in close proximity to the active areas containing the detecting agents on the surface of the sensor to facilitate binding between the detecting agent and target material (i.e., antigens/antibodies of the sample) if the target material is present in the sample. This reduces false negative results that may arise if diffusion alone is relied on. This is especially important feature to employ when testing samples where the target material may be present in very low concentrations.

Another aspect of the present disclosure is directed to a biosensor system. In one embodiment, the biosensor system is useful for characterizing a subject's immune response to pathogen exposure. In another embodiment, the biosensor system is useful for characterizing a pathogen's antigen profile. In either embodiment, the biosensor system comprises an electronic reader, where the electronic reader comprises a circuit for delivering a signal, and a processing device for reading the signal. The biosensor system further includes a biosensor as described herein that is operatively connected to the electronic reader and configured to receive the signal delivered by the circuit. The electronic reader is configured to deliver the signal to the biosensor and obtain an output impedance value before and after a sample has been applied to the array of active areas on the biosensor. The processing device is configured to compare the output impedance values to determine whether a binding event has occurred at one or more of the active areas to characterize the subject's immune response to pathogen exposure when the active areas contain pathogenic proteins/peptides, or to determine the presence of a pathogen in a sample and characterize its antigen profile when the active areas contain binding molecules (e.g., antibodies or antibody-based molecules). In some embodiments, the system is capable of characterizing both the subject's immune response and determine the presence and antigenic profile of a pathogen in a sample when the active areas across the biosensor contain binding molecules.

In some embodiments, the biosensor of the biosensor system comprises an electromagnet positioned beneath the substrate of the biosensor as described infra.

FIG. 10 is a schematic view of a biosensor system including an electronic reader 1038 for receiving the biosensor 1002. The electronic reader 1038 may include a slot 1040 for receiving the electrical connection 1044 of the biosensor 1002. Insertion of the biosensor 1002 completes a circuit within the electronic reader 1038 via the electrical connection 1044 comprising a plurality of electrical contacts, i.e., the drain bonding pads 1020, the shared source bonding pad 1028, and the gate bonding pad 1034. The electronic reader 1038 may further include a user interface 1042 for outputting information to a user. In some embodiments, the electronic reader 1038 may provide signals to a user interface not present on the actual reader 1038, e.g., via Bluetooth (or other communication means) to a monitor or other display.

In some embodiments, the biosensor systems as described herein further comprises a communication interface coupled to the electronic reader for transmitting data from the electronic reader, and a data management computing device configured to receive data from the electronic reader via the communication interface. In accordance with this embodiment, the data management computing device comprises a memory coupled to a processor which is configured to execute programmed instructions comprising and stored in the memory to geographically map immune response data to pathogen exposure and or pathogen antigenic profile data (i.e., the presence or evolution of various pathogen strains), based on data received from electronic reader.

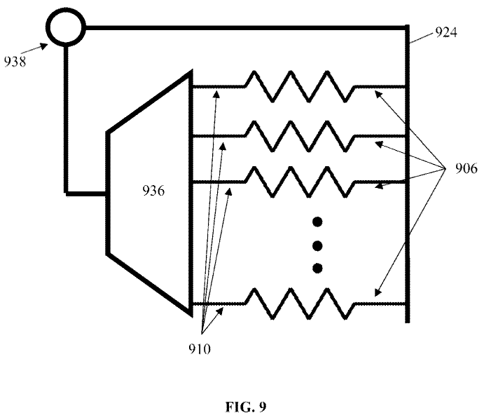

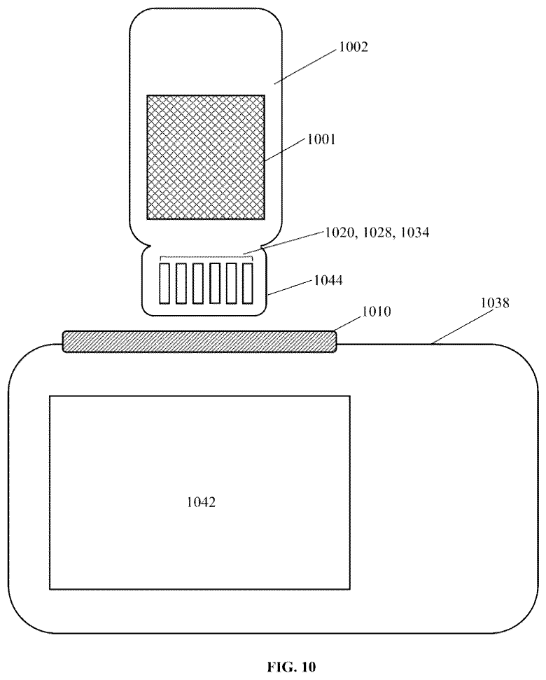

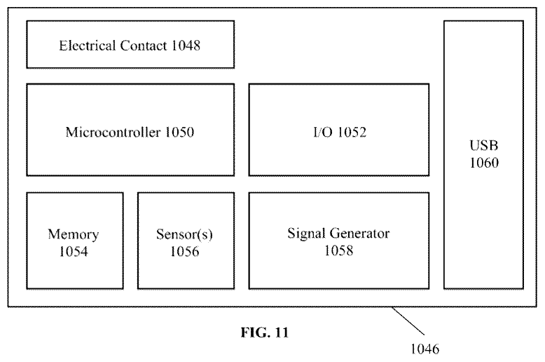

FIG. 11 is an exemplary embodiment of this aspect of the disclosure showing a block diagram of a circuit 1046, such as a circuit board with computing components for providing a signal to the biosensor 1002 and receiving a return signal to test a sample placed on the biosensor 1002. In an exemplary embodiment, the circuit 1046 includes a contact 1048 which may be an electrical contact for interacting with the electrical connection 1044 containing the electrical contacts 1020, 1028, 1034 of the biosensor 1002 to complete a circuit that includes the electronic reader 1038 and the biosensor 1002.

The circuit 1046 may also include computing components including, but not limited to, a microcontroller 1050, one or more I/O devices 1052, a memory or other storage component 1054, one or more sensors 1056, a signal generator 1058, and a USB or other communication hub 1060. The computing components are exemplary and may be replaced with other components to execute disclosed embodiments for testing a sample via the biosensor 1002.

The microcontroller or processor 1050 may be a processing device configured to monitor and control components of the circuit 1046, such as to perform setup, testing, and output processes via the electronic reader 1038. The processor 1050 may execute programmed instructions stored in the memory 1054 for any number of functions described and illustrated herein. The processor 1050 may include one or more central processing units (CPUs) or general purpose processors with one or more processing cores, for example, although other types of processor(s) can also be used.

The memory 1054 of the electronic reader 1038 stores these programmed instructions for aspect(s) of the present technology as described and illustrated herein, although some or all of the programmed instructions could be stored elsewhere. A variety of different types of memory storage devices, such as random access memory (RAM), read only memory (ROM), hard disk, solid state drives (SSD), flash memory, or other computer readable medium which is read from and written to by a magnetic, optical, or other reading and writing system that is coupled to the processor(s) 450, can be used for the memory 454.

The I/O device(s) 1052 may include the communication interface 1042, for example, to obtain input and provide output to and from a user. The communication interface 1042 of the electronic reader 1038 operatively couples and communicates between at least the electronic reader 1038 and an external computing device, in some embodiments, which are coupled together at least in part by one or more communication network(s) or a public cloud network. By way of example only, the communication network(s) can include local area network(s) (LAN(s)) or wide area network(s) (WAN(s)) and the public cloud network can include a WAN (e.g., the Internet). The communication network(s) and/or the public cloud network can use TCP/IP over Ethernet and industry-standard protocols, although other types or numbers of protocols or communication networks can be used. The communication network(s) and/or public cloud network in this example can employ any suitable interface mechanisms and network communication technologies including, for example, Ethernet-based Packet Data Networks (PDNs) and the like.

The sensors 1056 may include a voltage divider, resistance sensor, impedance sensor, or other device configured to determine a value associated with an electrical property at one or more locations on the biosensor 1002. The signal generator 1058 may be configured to generate an AC electrical signal for delivery to the biosensor 1002. The USB port 1060 may be a connection element for receiving and providing data exterior to the electronic reader.

The biosensor and biosensor systems described herein can be utilized to analyze a number of different biological samples to detect the presence of pathogen proteins and/or a subject's immune response to infection with a pathogenic organism or infectious agent. Accordingly, another aspect of the present disclosure is directed to a method of characterizing a subject's immune response to pathogen exposure. This method involves collecting a biological sample from a subject. A suitable sample is any biological fluid from the subject, including, without limitation, whole blood, blood serum, blood plasma, ascites fluid, cyst fluid, pleural fluid, peritoneal fluid, cerebrospinal fluid, tears, urine, saliva, sputum, lymph fluid, synovial fluid, amniotic fluid, follicular fluid, fluid of the respiratory, intestinal, and genitourinary trances.

The method further involves providing the biosensor system as described herein. Suitable biosensors include those containing a plurality of pathogen proteins and/or peptides immobilized at the active areas of the sensor. The method further involves delivering an electrical signal to the biosensor via the circuit of the electronic reader and determining a base resistance between the two or more signal electrodes at each active site on the biosensor. In some embodiments, the method further involves applying a control solution to at least one active area of the biosensor, such that the control solution is in operable contact with the carbon material between two signal electrodes and a gate electrode. In some embodiments, the signal received from the gate electrode can help determine the base resistance between the two or more signal electrodes at each active site. In some embodiments, the method further involves applying a first and a second control solution to two different areas at each active site on the biosensor. In some embodiments, each of the control solutions is in operable contact with the carbon material, at least two signal electrodes, and at least one gate electrode.

The method further involves applying the biological sample from the subject to the biosensor and identifying a change in the base resistance between the two or more signals electrodes at each active site on the biosensor resulting from said applying. In some embodiments, the biological sample is in operable contact with the carbon material and at least two signal electrodes. In some embodiments, the biological sample is in operable contact with the carbon material, at least two signal electrodes, and at least one gate electrode. The change in base resistance is indicative of an antibody from the sample binding to the immobilized pathogen proteins or peptides. The subject's immune response to pathogen exposure can be characterized based on the identified change in base resistance between the electrodes at the various active sites on the biosensor. Alternatively, the change in resistance is indicative of the presence of a pathogen in a sample, based on pathogen protein binding to the immobilized binding agents present in the active areas of the sensor. The pathogen's antigenic profile can be characterized based on the identified change in base resistance between the electrodes at the various active sites on the biosensor.

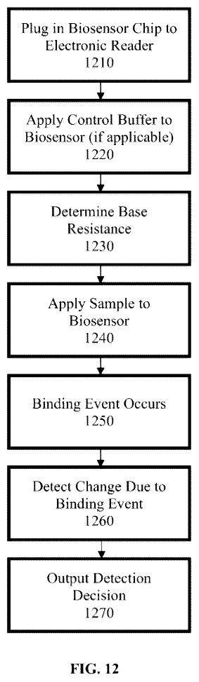

FIG. 12 provides an exemplary process 1200 for detecting a target moiety using a biosensor 1002 and electronic reader 1038. The biosensor 1002 is manufactured to include a collection of difference detecting agents as described supra.

An important feature of the biosensor device described herein relates to the arrangement of detecting agents across the sensing unit of the biosensor. In some embodiments, one or more active areas on the surface of the biosensor contains a collection of positive control detecting agents. Positive control agents include binding agents or proteins/peptides that are known to bind a component of the sample, either a naturally occurring substance in the sample or a substance that is introduced into the sample to facilitate the positive control detection. In addition, one or more active areas on the surface of the biosensor contain a collection of negative control detecting agents. Negative control detecting agents include binding agents or proteins/peptides that should not bind to any possible substance present in the sample. In addition, one or more active areas on the surface of the biosensor contain no detecting agents immobilized on the surface of the graphene. The presence of active areas containing positive control, negative control, and no detecting agents allows for accurate detection and relative quantitation of the presence of true target molecules (e.g., antibodies or antigens) in the test sample via differential signal detection between the active areas containing the control detecting agents and no detecting agent and the areas containing the detecting agents.

Another aspect of the present disclosure is directed to a method of characterizing a pathogen's antigen profile. This method involves collecting a pathogen containing sample and providing the biosensor system as disclosed herein. Suitable biosensors include those containing a collection of different binding molecules immobilized at the active areas of the sensor. The method further involves delivering an electrical signal to the biosensor via the circuit of the electronic reader and determining a base resistance between the two or more electrodes at each active site on the biosensor. The method further involves applying the biological sample from the subject to the biosensor and identifying a change in the base resistance between the two or more electrodes at each active site on the biosensor resulting from said applying. The change in base resistance is indicative of a pathogenic protein from the sample binding to the immobilized binding molecule. The presence of the pathogen in the sample and/or the pathogen's antigenic profile can be characterized based on the identified change in base resistance between the electrode as the various active sites on the biosensor.

In some embodiments, the biosensor of the system comprises an electromagnet positioned beneath the substrate of the biosensor. In accordance with this embodiment, the methods of characterizing a subject's immune response or a pathogen's antigenic profile as described herein further comprise labeling target material present in the collected biological sample with a magnetic moiety. In some embodiments, the target material in the sample is antibodies present in the sample. In some embodiments, the target material in the sample is pathogen proteins and/or peptides. In some embodiments, the target material in the sample is a mixture of both antibodies (produced by the host subject) and pathogen proteins (derived from the infectious agent infecting or having infected the host subject). Regardless, the target material, either antibodies and/or proteins are labeled with a magnetic moiety. The biological sample containing the labeled antibodies and/or proteins is mixed in a viscous fluid to create a viscous biological sample mixture for applying to the biosensor. Once the sample is applied, the electromagnet is turned on to localize the labeled antibodies and/or proteins of the biological sample mixture to the active areas on the surface of the substrate to facilitate binding between labeled antibodies and their cognate pathogenic proteins or peptides immobilized on the active surface, between labeled proteins and their cognate binding molecules immobilized on the active surface, or between both. After allowing sufficient time for binding between the immobilized detection agents and magnetically labeled target material, the electromagnet is turned off to release unbound labeled antibodies and/or protein prior to identifying a change in the base resistance between the two or more electrodes at each active site on the biosensor.

In some embodiments, labeling the target material (antibodies or proteins) in a sample involves contacting the biological sample with an azide containing magnetic moiety, and exposing the contacted sample with UV light to conjugate the magnetic moiety to antibodies within the biological sample. In some embodiments, the magnetic moiety is a magnetic bead. Suitable magnetic beads include, without limitation, ferrous oxide magnetic bead. Suitable magnetic beads have a diameter of 2 nm to 100 μm. For example, suitable magnetic beads have a diameter of 2 nm, 5 nm, 10 nm, 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, 1 μm, 5 μm, 10 μm, 20 μm, 30 μm, 40 μm, 50 μm, 60 μm, 70 μm, 80 μm, 90 μm, or 100 μm.

The viscous fluid of containing the magnetically labeled target material that is applied to the biosensor surface can comprise any viscous fluid. Suitable viscous fluids include, without limitation fluids comprising polyethylene glycol (PEG) or glycerin. In some embodiments the viscous fluid comprises about 20% PEG, about 25% PEG, about 30% PEG, about 35% PEG, about 40% PEG, about 45% PEG, about 50% PEG, about 55% PEG, about 60% PEG, about 65% PEG, about 70% PEG, about 75% PEG, about 80% PEG, about 85% PEG, or about 90% PEG. Any PEG known in the art is suitable for use in accordance with this aspect of the disclosure. In some embodiments, the PEG is PEG-400.

The following are non-limiting examples of embodiments described herein.
1. A biosensor comprising:
   an anti-static substrate comprising a planar surface;
   at least one spatially defined active area on the planar surface of the anti-static substrate, each active area comprising a carbon material, a first and a second signal electrode in operable contact with the carbon material, and at least one gate electrode;
   a plurality of detecting agents, wherein different detecting agents are positioned at different active areas and immobilized on the deposited carbon material of the active areas; and
   an electrical connection comprising a plurality of electrical contacts, each electrical contact configured to transmit an electrical signal between the first and second signal electrodes and at least one gate electrode of a single active area, and the electrical connection.
2. The biosensor of embodiment 1, wherein:
   the carbon material is deposited on the planar surface of the anti-static substrate,
   the first and the second signal electrodes are located on opposite sides of the carbon material and are covered by an insulating material, and
   the at least one gate electrode is located on top of the insulating material of either the first or the second signal electrodes.
3. The biosensor of embodiment 1, wherein:
   the first and second signal electrodes are deposited on the planar surface of the anti-static substrate, optionally with a bottom insulating material deposited on the planar surface of the anti-static substrate in between the first and the second signal electrodes,
   the carbon material is deposited on top of the first and the second signal electrodes, wherein the first and second signal electrodes are located on opposite sides of the carbon material,
   a top insulating material is deposited on top of the carbon material on opposite sides of the carbon material and approximately above the first and the second signal electrodes, and
   the at least one gate electrode is located on top of the top insulating material above either the first or the second signal electrodes.
4. The biosensor of any one of embodiments 1-3, wherein the spatially defined array of active areas comprises at least 2 active areas.
5. The biosensor of any one of embodiments 1-4, wherein the carbon material is graphene, carbon nanotube, or a combination thereof.
6. The biosensor of any one of embodiments 1-5, wherein the at least two signal electrodes and/or gate electrodes comprise a conductive metal selected from Ti, Cu, Ag, Ir, Pt, Au, or any combination or oxide thereof.
7. The biosensor of any one of embodiments 1-5, wherein the at least two signal electrodes and/or gate electrodes comprise a carbon-based conducting material selected from carbon nanotubes, graphene oxide, or any combination thereof.
8. The biosensor of any one of embodiments 1-7, wherein each active area further comprises a preservative solution.
9. The biosensor of any one of embodiments 1-8, wherein each detecting agent is immobilized on the deposited carbon material via a hydrophobic linker, wherein said hydrophobic linker is coupled to a binding molecule via the detecting agent's amino or carboxy terminus.
10. The biosensor of embodiment 9, wherein the hydrophobic linker is a peptide linker comprising two or more linker amino acid residues and one or more aromatic amino acid residues
11. The biosensor of embodiment 10, wherein the two or more linker amino acid residues are selected from glycine, alanine, serine, and combinations thereof.
12. The biosensor of embodiment 11, wherein the hydrophobic linker comprises a polycyclic aromatic hydrocarbon.
13. The biosensor of any one of embodiments 1-12, wherein plurality of detecting agents comprise pathogen proteins or peptides thereof, binding molecules capable of binding pathogen proteins of peptides thereof, polynucleotides, or combinations thereof.
14. The biosensor of embodiment 13, wherein said biosensor further comprises a collection of antibody mimetics, aptamers, DNA molecules, RNA molecules, modified oligonucleotides, or a combination thereof, wherein different members of the collection bind different pathogen proteins and wherein different members of the collection are positioned at different active areas not occupied by the detecting agents, and wherein said members of the collection are immobilized on the deposited carbon material of said active areas.
15. The biosensor of any one of embodiments 13 or 14, wherein the pathogen is one or more infectious agents selected from a virus, a bacterium, a toxin, or a combination thereof.
16. The biosensor of embodiment 15, wherein the pathogen is one or more viruses selected from SARS-COV-2, Influenza A, Influenza B, Human papilloma virus, Venezuelan equine encephalitis virus, Vaccinia virus, Ebola virus, Lassa fever virus, Rift Valley fever virus and combinations thereof.
17. The biosensor of embodiment 15, wherein the pathogen is one or more bacteria selected from the group consisting of Pseudomonas aeruginosa, Neisseria gonorrhoeae, Chlamydia trachomatis, Treponema pallidum, Bacillus anthracis, Yersinia pestis, Francisella tularensis, Burkholderia pseudomallei, Burkholderia mallei, and combinations thereof.
18. The biosensor of embodiment 15, wherein the pathogen is one or more toxins selected from Ricin toxin, Botulinum Toxin A/B/E, Staphylococcus enterotoxin B (SEB), Abrin toxin, T-2 toxin, B. anthracis LF toxin, B. anthracis EF toxin, B. anthracis PA toxin, and combinations thereof.
19. The biosensor of any one of embodiments 13-18, wherein each of the plurality of pathogen peptides is between 5 and 50 amino acid residues in length.
20. The biosensor of any one of embodiments 13-19, wherein the binding molecules of the collection are antibody-based molecules.
21. The biosensor of embodiment 20, wherein the antibody-based molecules are selected from antibodies, epitope-binding domains thereof, antibody derivatives, antibody mimetics, or combinations thereof.
22. The biosensor of embodiment 21, wherein the antibody mimetics are selected from the group consisting of an affibodies, affilins, affimers, monobodies, and DARPINs.
23. The biosensor of any one of embodiments 1-22 further comprising an electromagnet positioned beneath the substrate of the biosensor.
24. The biosensor of any one of embodiments 1-23, wherein the anti-static substrate comprises a single-layer of anti-static polymeric material.
25. The biosensor of any one of embodiments 1-24, wherein the anti-static substrate comprises a polymeric material with an anti-static additive.
26. The biosensor of any one of embodiments 1-25, wherein the anti-static substrate comprises an anti-static printed circuit board.
27. A biosensor system for characterizing a subject's immune response to pathogen exposure, the system comprising:
   an electronic reader comprising:
      a circuit for delivering a signal; and
      a processing device for reading the signal;
   a biosensor of any one of embodiments 1-26 operatively connected to the electronic reader via the electrical connection of the biosensor and configured to receive the signal delivered by the circuit;
   wherein the electronic reader is configured to deliver the signal to the biosensor and obtain an output impedance value before and after a sample has been applied to the array of active areas on the biosensor, and said processing device is configured to compare the output impedance values to determine whether a binding event has occurred at one or more of the active areas to characterize the subject's immune response to pathogen exposure.
28. The biosensor system of embodiment 27, wherein the biosensor comprises an electromagnet positioned beneath the substrate of the biosensor.
29. The biosensor system of embodiments 27 or 28, further comprising:
   a communication interface coupled to the electronic reader for transmitting data from the electronic reader;
   a data management computing device configured to receive data from the electronic reader via the communication interface, said data management computing device comprising a memory coupled to a processor which is configured to execute programmed instructions comprising and stored in the memory to geographically map immune response data to pathogen exposure, based on data received from electronic reader.
30. A method of characterizing a subject's immune response to pathogen exposure, said method comprising:
   collecting a biological sample from a subject;
   providing the biosensor system of any one of embodiments 27-29;
   delivering an electrical signal to the biosensor via the circuit of the electronic reader;
   determining a base resistance between the first and second signal electrodes at each active area on the biosensor;
   applying the biological sample from the subject to at least one active area on the biosensor, such that the biological sample is in operable contact with the carbon material between the first and second signal electrodes, and the at least one gate electrode at the at least one active area;
   identifying a change in the base resistance between the first and second signal electrodes, resulting from applying the biological sample to the at least one active area; and
   characterizing the subject's immune response to the pathogen, or the pathogen's antigen profile based on the change in the base resistance between the first and second signal electrodes at the at least one active area.
31. The method of embodiment 30, wherein the biosensor of the system comprises an electromagnet positioned beneath the substrate of the biosensor, said method further comprising:
   labeling, after said collecting, antibodies present in the biological sample with a magnetic moiety;
   mixing the biological sample containing the labeled antibodies with a viscous fluid to create a viscous biological sample mixture for said applying;
   turning on said electromagnet to localize the labeled antibodies of the biological sample mixture to the active areas on the surface of the substrate during said applying to facilitate binding between labeled antibodies and their cognate detecting agents immobilized on the active surface; and
   turning off said electromagnet to release unbound labeled antibodies prior to said identifying.
32. The method of embodiment 31, wherein said labeling comprises:
   contacting the biological sample with an azide containing magnetic moiety, and
   exposing the contacted sample with UV light to conjugate the magnetic moiety to antibodies within the biological sample.
33. The method of embodiment 32, wherein the magnetic moiety is a magnetic bead.
34. The method of embodiment 33, wherein the magnetic bead is a ferrous oxide magnetic bead.
35. The method of embodiment 33, wherein the magnetic bead has a diameter of 2 nm to 100 μm.
36. The method of embodiment 31, wherein the viscous fluid comprises polyethylene glycol (PEG) or glycerin.
37. The method of embodiment 36, wherein the PEG is PEG-400.
38. The method of embodiments 36 or 37, wherein the viscous fluid comprises about 20% to about 90% PEG.
39. A biosensor comprising:
   an anti-static substrate comprising a planar surface;
   at least one spatially defined active area on the planar surface of the anti-static substrate, each active area comprising a carbon material, a first, second, and a third signal electrode in operable contact with the carbon material, and at least one gate electrode;
   a plurality of detecting agents, wherein different detecting agents are positioned at different active areas and immobilized on the deposited carbon material of the active areas; and
   an electrical connection comprising a plurality of electrical contacts, each electrical contact configured to transmit an electrical signal between the first and second signal electrodes and at least one gate electrode of a single active area, and the electrical connection.
40 The biosensor of embodiment 39, wherein:
   the carbon material is deposited on the planar surface of the anti-static substrate,
   the first and the second signal electrodes are located on opposite sides of the carbon material and are covered by an insulating material,
   the third signal electrode is located on top of the carbon material, between from the first and the second signal electrodes, and is surrounded by an insulating material, and
   the at least one gate electrode is located on top of the insulating material of the first signal electrode.
41. The biosensor of embodiment 39, wherein:
   the first, second, and third signal electrodes are deposited on the planar surface of the anti-static substrate, optionally with a bottom insulating material deposited on the planar surface of the anti-static substrate in between the first, second, and third signal electrodes,
   the carbon material is deposited on top of the first, second, and third signal electrodes, wherein the first and second signal electrodes are located on opposite sides of the carbon material, and the third signal electrode is located between from the first and the second signal electrodes,
   a top insulating material is deposited on top of the carbon material, approximately above the first, second, and third signal electrodes, and
   the at least one gate electrode is located on top of the top insulating material above the first signal electrode.
42. The biosensor of any one of embodiments 39-41, wherein the spatially defined array of active areas comprises at least 2 active areas.
43. The biosensor of any one of embodiments 39-42, wherein the carbon material is graphene, carbon nanotube, or a combination thereof.
44. The biosensor of any one of embodiments 39-43, wherein the at least two signal electrodes and/or gate electrodes comprise a conductive metal selected from Ti, Cu, Ag, Ir, Pt, Au, or any combination or oxide thereof.
45. The biosensor of any one of embodiments 39-43, wherein the at least two signal electrodes and/or gate electrodes comprise a carbon-based conducting material selected from carbon nanotubes, graphene oxide, or any combination thereof.
46. The biosensor of any one of embodiments 39-45, wherein each active area further comprises a preservative solution.
47. The biosensor of any one of embodiments 39-46, wherein each detecting agent is immobilized on the deposited carbon material via a hydrophobic linker, wherein said hydrophobic linker is coupled to a binding molecule via the detecting agent's amino or carboxy terminus.
48. The biosensor of embodiment 47, wherein the hydrophobic linker is a peptide linker comprising two or more linker amino acid residues and one or more aromatic amino acid residues
49. The biosensor of embodiment 48, wherein the two or more linker amino acid residues are selected from glycine, alanine, serine, and combinations thereof.
50. The biosensor of embodiment 49, wherein the hydrophobic linker comprises a polycyclic aromatic hydrocarbon.
51. The biosensor of any one of embodiments 39-50, wherein plurality of detecting agents comprise pathogen proteins or peptides thereof, binding molecules capable of binding pathogen proteins of peptides thereof, polynucleotides, or combinations thereof.
52. The biosensor of embodiment 51, wherein said biosensor further comprises a collection of antibody mimetics, aptamers, DNA molecules, RNA molecules, modified oligonucleotides, or a combination thereof, wherein different members of the collection bind different pathogen proteins and wherein different members of the collection are positioned at different active areas not occupied by the detecting agents, and wherein said members of the collection are immobilized on the deposited carbon material of said active areas.
53. The biosensor of any one of embodiments 51 or 52, wherein the pathogen is one or more infectious agents selected from a virus, a bacterium, a toxin, or a combination thereof.
54. The biosensor of embodiment 53, wherein the pathogen is one or more viruses selected from SARS-COV-2, Influenza A, Influenza B, Human papilloma virus, Venezuelan equine encephalitis virus, Vaccinia virus, Ebola virus, Lassa fever virus, Rift Valley fever virus and combinations thereof.
55. The biosensor of embodiment 53, wherein the pathogen is one or more bacteria selected from the group consisting of Pseudomonas aeruginosa, Neisseria gonorrhoeae, Chlamydia trachomatis, Treponema pallidum, Bacillus anthracis, Yersinia pestis, Francisella tularensis, Burkholderia pseudomallei, Burkholderia mallei, and combinations thereof.
56. The biosensor of embodiment 53, wherein the pathogen is one or more toxins selected from Ricin toxin, Botulinum Toxin A/B/E, Staphylococcus enterotoxin B (SEB), Abrin toxin, T-2 toxin, B. anthracis LF toxin, B. anthracis EF toxin, B. anthracis PA toxin, and combinations thereof.
57. The biosensor of any one of embodiments 51-56, wherein each of the plurality of pathogen peptides is between 5 and 50 amino acid residues in length.
58. The biosensor of any one of embodiments 51-57, wherein the binding molecules of the collection are antibody-based molecules.
59. The biosensor of embodiment 58, wherein the antibody-based molecules are selected from antibodies, epitope-binding domains thereof, antibody derivatives, antibody mimetics, or combinations thereof.
60. The biosensor of embodiment 59, wherein the antibody mimetics are selected from the group consisting of an affibodies, affilins, affimers, monobodies, and DARPINs.
61. The biosensor of any one of embodiments 39-60 further comprising an electromagnet positioned beneath the substrate of the biosensor.
62. The biosensor of any one of embodiments 39-61, wherein the anti-static substrate comprises a single-layer of anti-static polymeric material.
63. The biosensor of any one of embodiments 39-62, wherein the anti-static substrate comprises a polymeric material with an anti-static additive.
64. The biosensor of any one of embodiments 39-63, wherein the anti-static substrate comprises an anti-static printed circuit board.
65. A biosensor system for characterizing a subject's immune response to pathogen exposure, the system comprising:
   an electronic reader comprising:
      a circuit for delivering a signal; and
      a processing device for reading the signal;
   a biosensor of any one of embodiments 39-64 operatively connected to the electronic reader via the electrical connection of the biosensor and configured to receive the signal delivered by the circuit;
   wherein the electronic reader is configured to deliver the signal to the biosensor and obtain an output impedance value before and after a sample has been applied to the array of active areas on the biosensor, and said processing device is configured to compare the output impedance values to determine whether a binding event has occurred at one or more of the active areas to characterize the subject's immune response to pathogen exposure.
66. The biosensor system of embodiment 65, wherein the biosensor comprises an electromagnet positioned beneath the substrate of the biosensor.
67. The biosensor system of embodiments 65 or 66, further comprising:
   a communication interface coupled to the electronic reader for transmitting data from the electronic reader;
   a data management computing device configured to receive data from the electronic reader via the communication interface, said data management computing device comprising a memory coupled to a processor which is configured to execute programmed instructions comprising and stored in the memory to geographically map immune response data to pathogen exposure, based on data received from electronic reader.
68. A method of characterizing a subject's immune response to pathogen exposure, said method comprising:
   collecting a biological sample from a subject;
   providing the biosensor system of any one of embodiments 65-67;
   delivering an electrical signal to the biosensor via the circuit of the electronic reader;
   applying a control solution to at least one active area on the biosensor, such that the control solution is in operable contact with the carbon material between the first and the third signal electrodes, and the at least one gate electrode at the at least one active area;
   determining a base resistance between the second and third signal electrodes at the at least one active area;
   applying the biological sample from the subject to the at least one active area, such that the biological sample is in operable contact with the carbon material between the second and third signal electrodes;
   identifying a change in the base resistance between the second and third signal electrodes resulting from applying the biological sample to the at least one active area; and
   characterizing the subject's immune response to the pathogen, or the pathogen's antigen profile based on the change in the base resistance between the second and third signal electrodes at the at least one active area.
69. The method of embodiment 68, wherein the biosensor of the system comprises an electromagnet positioned beneath the substrate of the biosensor, said method further comprising:
   labeling, after said collecting, antibodies present in the biological sample with a magnetic moiety;
   mixing the biological sample containing the labeled antibodies with a viscous fluid to create a viscous biological sample mixture for said applying;
   turning on said electromagnet to localize the labeled antibodies of the biological sample mixture to the active areas on the surface of the substrate during said applying to facilitate binding between labeled antibodies and their cognate detecting agents immobilized on the active surface; and turning off said electromagnet to release unbound labeled antibodies prior to said identifying.
70. The method of embodiment 69, wherein said labeling comprises:
   contacting the biological sample with an azide containing magnetic moiety, and
   exposing the contacted sample with UV light to conjugate the magnetic moiety to antibodies within the biological sample.
71. The method of embodiment 70, wherein the magnetic moiety is a magnetic bead.
72. The method of embodiment 71, wherein the magnetic bead is a ferrous oxide magnetic bead.
73. The method of embodiment 71, wherein the magnetic bead has a diameter of 2 nm to 100 μm.
74. The method of embodiment 69, wherein the viscous fluid comprises polyethylene glycol (PEG) or glycerin.
75. The method of embodiment 74, wherein the PEG is PEG-400.
76. The method of embodiments 74 or 75, wherein the viscous fluid comprises about 20% to about 90% PEG.
77. A biosensor comprising:
   an anti-static substrate comprising a planar surface;
   at least one spatially defined active area on the planar surface of the anti-static substrate, each active area comprising a carbon material, a first, a second, a third, and a fourth signal electrode in operable contact with the carbon material, and at least a first and a second gate electrode;
   a plurality of detecting agents, wherein different detecting agents are positioned at different active areas and immobilized on the deposited carbon material of the active areas; and
   an electrical connection comprising a plurality of electrical contacts, each electrical contact configured to transmit an electrical signal between the first, second, third, and fourth signal electrodes and the at least a first and a second gate electrodes of a single active area, and the electrical connection.
78. The biosensor of embodiment 77, wherein:
   the carbon material is deposited on the planar surface of the anti-static substrate,
   the first and the second signal electrodes are located on opposite sides of the carbon material and are covered by an insulating material,
   the third signal electrode is located on top of the carbon material, between the first and the fourth signal electrodes, and is surrounded by an insulating material,
   the fourth signal electrode is located on top of the carbon material, between the second and the third signal electrodes, and is surrounded by an insulating material,
   the at least a first gate electrode is located on top of the insulating material of the first signal electrode, and
   the at least a second gate electrode is located on top of the insulating material of the second signal electrode.
79. The biosensor of embodiment 77, wherein:
   the first, second, third, and fourth signal electrodes are deposited on the planar surface of the anti-static substrate, optionally with a bottom insulating material deposited on the planar surface of the anti-static substrate in between the first, second, third, and fourth signal electrodes,
   the carbon material is deposited on top of the first, second, third, and fourth signal electrodes, wherein the first and second signal electrodes are located on opposite sides of the carbon material, the third signal electrode is located between the first and the fourth signal electrodes, and the fourth signal electrode is located between the second and the third signal electrodes,
   a top insulating material is deposited on top of the carbon material, approximately above the first, second, third, and fourth signal electrodes,
   the at least a first gate electrode is located on top of the top insulating material approximately above the first signal electrode, and
   the at least a second gate electrode is located on top of the top insulating material approximately above the second signal electrode.
80. The biosensor of any one of embodiments 77-79, wherein the spatially defined array of active areas comprises at least 2 active areas.
81. The biosensor of any one of embodiments 77-80, wherein the carbon material is graphene, carbon nanotube, or a combination thereof.
82. The biosensor of any one of embodiments 77-81, wherein the at least two signal electrodes and/or gate electrodes comprise a conductive metal selected from Ti, Cu, Ag, Ir, Pt, Au, or any combination or oxide thereof.
83. The biosensor of any one of embodiments 77-81, wherein the at least two signal electrodes and/or gate electrodes comprise a carbon-based conducting material selected from carbon nanotubes, graphene oxide, or any combination thereof.
84. The biosensor of any one of embodiments 77-83, wherein each active area further comprises a preservative solution.
85. The biosensor of any one of embodiments 77-84, wherein each detecting agent is immobilized on the deposited carbon material via a hydrophobic linker, wherein said hydrophobic linker is coupled to a binding molecule via the detecting agent's amino or carboxy terminus.
86. The biosensor of embodiment 85, wherein the hydrophobic linker is a peptide linker comprising two or more linker amino acid residues and one or more aromatic amino acid residues 87. The biosensor of embodiment 86, wherein the two or more linker amino acid residues are selected from glycine, alanine, serine, and combinations thereof.
88. The biosensor of embodiment 87, wherein the hydrophobic linker comprises a polycyclic aromatic hydrocarbon.
89. The biosensor of any one of embodiments 77-88, wherein plurality of detecting agents comprise pathogen proteins or peptides thereof, binding molecules capable of binding pathogen proteins of peptides thereof, polynucleotides, or combinations thereof.
90. The biosensor of embodiment 89, wherein said biosensor further comprises a collection of antibody mimetics, aptamers, DNA molecules, RNA molecules, modified oligonucleotides, or a combination thereof, wherein different members of the collection bind different pathogen proteins and wherein different members of the collection are positioned at different active areas not occupied by the detecting agents, and wherein said members of the collection are immobilized on the deposited carbon material of said active areas.
91. The biosensor of any one of embodiments 89 or 90, wherein the pathogen is one or more infectious agents selected from a virus, a bacterium, a toxin, or a combination thereof.
92. The biosensor of embodiment 91, wherein the pathogen is one or more viruses selected from SARS-COV-2, Influenza A, Influenza B, Human papilloma virus, Venezuelan equine encephalitis virus, Vaccinia virus, Ebola virus, Lassa fever virus, Rift Valley fever virus and combinations thereof.
93. The biosensor of embodiment 91, wherein the pathogen is one or more bacteria selected from the group consisting of Pseudomonas aeruginosa, Neisseria gonorrhoeae, Chlamydia trachomatis, Treponema pallidum, Bacillus anthracis, Yersinia pestis, Francisella tularensis, Burkholderia pseudomallei, Burkholderia mallei, and combinations thereof.
94. The biosensor of embodiment 91, wherein the pathogen is one or more toxins selected from Ricin toxin, Botulinum Toxin A/B/E, Staphylococcus enterotoxin B (SEB), Abrin toxin, T-2 toxin, B. anthracis LF toxin, B. anthracis EF toxin, B. anthracis PA toxin, and combinations thereof.
95. The biosensor of any one of embodiments 90-94, wherein each of the plurality of pathogen peptides is between 5 and 50 amino acid residues in length.
96. The biosensor of any one of embodiments 90-95, wherein the binding molecules of the collection are antibody-based molecules.
97. The biosensor of embodiment 96, wherein the antibody-based molecules are selected from antibodies, epitope-binding domains thereof, antibody derivatives, antibody mimetics, or combinations thereof.
98. The biosensor of embodiment 97, wherein the antibody mimetics are selected from the group consisting of an affibodies, affilins, affimers, monobodies, and DARPINs.
99. The biosensor of any one of embodiments 77-98 further comprising an electromagnet positioned beneath the substrate of the biosensor.
100. The biosensor of any one of embodiments 77-99, wherein the anti-static substrate comprises a single-layer of anti-static polymeric material.
101. The biosensor of any one of embodiments 77-100, wherein the anti-static substrate comprises a polymeric material with an anti-static additive.
102. The biosensor of any one of embodiments 77-101, wherein the anti-static substrate comprises an anti-static printed circuit board.
103. A biosensor system for characterizing a subject's immune response to pathogen exposure, the system comprising:
   an electronic reader comprising:
      a circuit for delivering a signal; and
      a processing device for reading the signal;
   a biosensor of any one of embodiments 77-102 operatively connected to the electronic reader via the electrical connection of the biosensor and configured to receive the signal delivered by the circuit;
   wherein the electronic reader is configured to deliver the signal to the biosensor and obtain an output impedance value before and after a sample has been applied to the array of active areas on the biosensor, and said processing device is configured to compare the output impedance values to determine whether a binding event has occurred at one or more of the active areas to characterize the subject's immune response to pathogen exposure.
104. The biosensor system of embodiment 103, wherein the biosensor comprises an electromagnet positioned beneath the substrate of the biosensor.
105. The biosensor system of embodiments 103 or 104, further comprising:
   a communication interface coupled to the electronic reader for transmitting data from the electronic reader;
   a data management computing device configured to receive data from the electronic reader via the communication interface, said data management computing device comprising a memory coupled to a processor which is configured to execute programmed instructions comprising and stored in the memory to geographically map immune response data to pathogen exposure, based on data received from electronic reader.
106. A method of characterizing a subject's immune response to pathogen exposure, said method comprising:
   collecting a biological sample from a subject;
   providing the biosensor system of any one of embodiments 103-105;
   delivering an electrical signal to the biosensor via the circuit of the electronic reader;
   applying a first control solution to at least one active area on the biosensor, such that the first control solution is in operable contact with the carbon material between the first and the third signal electrodes, and the first gate electrode at the at least one active area;
   applying a second control solution to the at least one active area, such that the second control solution is in operable contact with the carbon material between the second and the fourth signal electrodes, and the second gate electrode at the at least one active area;
   determining a base resistance between the third and fourth signal electrodes at the at least one active area;
   applying the biological sample from the subject to the at least one active area, such that the biological sample is in operable contact with the carbon material between the second and third signal electrodes;
   identifying a change in the base resistance between the second and third signal electrodes resulting from applying the biological sample to the at least one active area; and
   characterizing the subject's immune response to the pathogen, or the pathogen's antigen profile based on the change in the base resistance between the second and third signal electrodes at the at least one active area.
107. The method of embodiment 106, wherein the biosensor of the system comprises an electromagnet positioned beneath the substrate of the biosensor, said method further comprising:
   labeling, after said collecting, antibodies present in the biological sample with a magnetic moiety;
   mixing the biological sample containing the labeled antibodies with a viscous fluid to create a viscous biological sample mixture for said applying;
   turning on said electromagnet to localize the labeled antibodies of the biological sample mixture to the active areas on the surface of the substrate during said applying to facilitate binding between labeled antibodies and their cognate detecting agents immobilized on the active surface; and
   turning off said electromagnet to release unbound labeled antibodies prior to said identifying.
108. The method of embodiment 107, wherein said labeling comprises:
   contacting the biological sample with an azide containing magnetic moiety, and
   exposing the contacted sample with UV light to conjugate the magnetic moiety to antibodies within the biological sample.
109. The method of embodiment 108, wherein the magnetic moiety is a magnetic bead.
110. The method of embodiment 109, wherein the magnetic bead is a ferrous oxide magnetic bead.
111. The method of embodiment 109, wherein the magnetic bead has a diameter of 2 nm to 100 μm.
112. The method of embodiment 107, wherein the viscous fluid comprises polyethylene glycol (PEG) or glycerin.
113. The method of embodiment 112, wherein the PEG is PEG-400.
114. The method of embodiments 112 or 113, wherein the viscous fluid comprises about 20% to about 90% PEG.
115. A method of making the biosensor of any one of embodiments 1-26, 39-64, or 77-102, wherein the method comprising depositing a graphene material on the surface of the biosensor using a contour ablation process.
116. A method of making a biosensor, the method comprising:
   obtaining a substrate with at least two layers, wherein there is a z-height difference between the at least two layers,
   transferring graphene onto the substrate, wherein the graphene breaks along the at least two layers due to the z-height difference,
   washing the biosensor to remove excess graphene,
   and, optionally, adding additional layers to the biosensor.
117. The method of embodiment 116, wherein the z-height difference between the at least two layers is about 50 nanometers to about 3 millimeters.
118. The method of embodiment 116 or 117, wherein the substrate comprises at least 3 layers.
119. The method of embodiment 116 or 117, wherein the substrate comprises at least 4 layers.
120. The method of any one of embodiments 116-119, wherein the additional layers comprise insulating material, or electrode material, or both.

### EXAMPLES

The examples below are intended to exemplify the practice of embodiments of the disclosure but are by no means intended to limit the scope thereof.

### Example 1: Comparison of Biologic Adhesion and Target Incubation on Graphene Silicon Sensor and Graphene Polyethylene Terephthalate (PET) Sensor Chips

To use these graphene surfaces as a diagnostic platform, the antibody or protein of interest is first adhered. This process (referred to herein as “adhesion”) starts with immobilizing a pyrene molecule to the surface of the graphene. Pyrene and graphene form a strong interaction through-T stacking of the sp² carbons in each respective ring structure. Then each circuit undergoes a seven-step adhesion process as described in Goldsmith et al., “Digital Biosensing by Foundry-Fabricated Graphene Sensors,” Sci. Rep. 9:434 (2019), which is hereby incorporated by reference in its entirety. This process involves activating the pyrene and then covalently attaching the protein or antibody of interest to the pyrene. The surface is then blocked and the reaction quenched by PEG-amine and ethanolamine, respectively. Following adhesion, the chips undergo washing prior to target incubation. In this portion of the experiment, the chips are calibrated by the addition of PBS buffer (a critical step for data normalization) followed by the addition of the antibody or protein of interest.

### Graphene Silicon Chips (Comparator)

Graphenea Silicon Chips (Graphena Chips) are individual graphene circuits purchased from Graphenea (Cambridge, MA). Each chip contains 12 circuits which can be used for multiplexing.

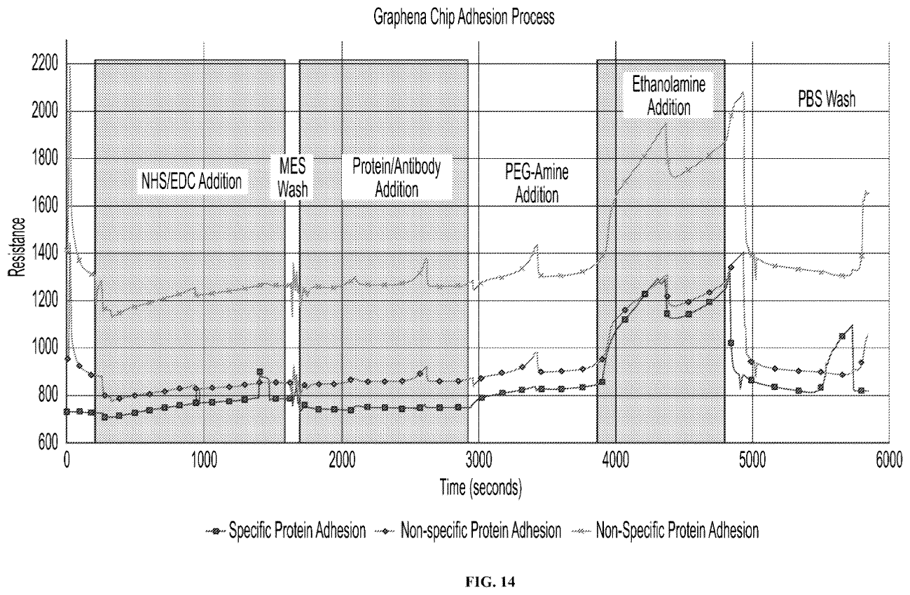

Adhesion: The adhesion process for BSA or Spike 1 protein on a Graphena chip followed the seven-step process described above. A graph of circuit resistance over the 7-step adhesion process is shown in FIG. 14. Small jumps in the data occur when buffer is added to maintain surface saturation and are considered negligible when observing the entire protein adhesion process. Unfortunately, this method does not allow one to quantify the extent of protein or antibody surface adhesion. However, multiplexing is possible on these chips, which allows for the adhesion of multiple positive and negative controls in a single run. Additionally, Graphenea chips consistently show low standard deviations among circuits of the same run, providing confidence in the overall reliability of these circuits.
Target Incubation on the Graphenea Chips. Graphenea chips typically have low overall noise and a particularly good signal to noise ratio during the target incubation experiments. Specific Binding to the adhered BSA was tested with the addition of a BSA Antibody (1 mg/mL) to the circuit. Nonspecific Binding to the adhered Spike 1 protein was tested with the addition of BSA Antibody (1 mg/mL) to the circuit. The Blank Control is an unlabeled circuit with BSA Antibody (1 mg/mL) added to the circuit. The results, as summarized in Table 1 below, show a specific binding, non-specific binding, and blank control experiment conducted on one chip through multiplexing. This data is normalized using the PBS addition at −300 seconds in order to remove the effects of drift and accurately compare data. From this trial, selective binding is observed.

TABLE 1
Target Incubation on a Graphenea Chip.

| Binding type | Normalized Resistance |
|---|---|
| Specific Binding | 400 ± 20 Ohms |
| Non-Specific Binding | 240 ± 30 Ohms |
| Blank Control | 150 ± 30 Ohms |

### General Graphene PET-Graphene Sheets

General Graphene PET-Graphene Sheets (PET-Graphene) were purchased as 4.5″×3.8″ sheets of PET plastic coated with a single layer of graphene. These sheets were cut into 0.7″×0.3″ rectangles and spotted with silver paint to establish electrical connection across the circuit.
Adhesion: Experiments using a simplified 3 step adhesion process (eliminating blocking and quenching steps) confirmed the presence of adhered antibodies (BSA or pseudomonas) or protein using AFM and SEM on a silicon wafer. Using this three-step process on the PET-Graphene, the presence of protein was confirmed using a Coomassie Blue stain. Results from this adhesion process yields bound protein that was used for the following target incubation experiments.
Target Incubation on the PET-Graphene Chips: Target incubation in PET-Graphene initially showed promising results (see results in Table 2 below). There was a dose-response in the specific binding samples. Specific Binding (2×) to the adhered BSA antibody was tested with the addition of BSA protein (14 μg/mL) to the circuit. Specific Binding (1×) to the adhered BSA antibody was tested with the addition of BSA protein (7 μg/mL) to the circuit. Nonspecific Binding to the adhered Pseudomonas antibody with the addition of BSA protein (7 μg/mL) to the circuit. The Blank Control had nothing adhered (i.e., an unlabeled circuit) with BSA antibody (1 mg/mL) added to the circuit.

TABLE 2
Target Incubation with PET-Graphene Chips.

| Binding type | Normalized Resistance |
|---|---|
| Specific Binding (2X) | 3.5 ± 0.2 Ohms |
| Specific Binding (1X) | 2.9 ± 0.2 Ohms |
| Non-Specific Binding | 2.1 ± 0.3 Ohms |
| Blank | 1.3 ± 0.3 Ohms |

### Example 2: Electromagnetic Substrate Enhanced Target

Overview: As described herein the biosensors of the disclosure can contain an electromagnetic that is position beneath the biosensor surface. This electromagnet comprises an on/off switch to allow users to control the diffusion of the sample components, which have been magnetically labeled to the surface of the biosensor. The method of use generally involves using UV or chemical activation as known in the art to conjugate antibodies and/or proteins in a biological sample to magnetic moieties, such as magnetic beads. The labeled sample is mixed with a high-density fluid to suspend magnetic bead-protein complex in solution and the mixture is added to the biosensor. FIG. 13A is a schematic showing the magnetically labeled target components of the sample applied to the biosensor when the electromagnet, positioned beneath the surface of the sensor is turned off.

When the electromagnet is turned on the magnetically labeled proteins and antibodies in the sample are brought in close proximity to the surface and active areas containing the detecting agents thereof as shown in FIG. 13B. This allows for target material in the sample to bind to its cognate binding partner (either an antibody, antibody-based molecule, or protein/peptide immobilized on the surface of the sensor).

Once time allowed for any binding interactions to occur has passed, the electromagnet is turned off to release the unbound magnetically labeled target material back into solution as shown in FIG. 13C below. The target material that is specifically bound to its immobilized binding partner on the surface remains bound to the surface, and a change in electrical current between circuits resulting from the binding of the target material specifically to its immobilized binding partner on the surface is measured.
Experimental Analysis: Blank circuits (die no. 56 on GG2) were rehydrated with 2.5 μL 80% PEG-400, 0.01×PBS. PEG-400 provides viscosity to the solution, which limits water evaporation (and therefore signal drift) and allows to probe binding kinetics (by slowing particle diffusion). Readings were obtained from two circuits. Two sets of ground magnets were placed under the wafer with the analyzed die centered on the magnet.

After initiation of the experiment, a steep decrease in signal is observed from 0-200 seconds which is likely due to signal stabilization (FIG. 15, far left box). Baseline signal is recorded from 200-600 seconds, with an average slope of 0.038. At 594 seconds, 2.5 μL of 2 nm-ferrous oxide magnetic beads conjugated to BSA in 0.01×PBS were added for a final concentration of 40% PEG-400, approx. 0.12 mg/mL BSA, 0.01×PBS. This addition led to an increase in signal from 600-800 seconds, with a slope of 1.09 (see FIG. 15, middle box). This transition stabilized at 800 seconds and baselined with a ΔV of approximately 200 mV until 1200 seconds (slope=0.0057). At 1188 seconds a second addition of 2.5 μL of 2 nm-ferrous oxide magnetic beads conjugated to BSA in 0.01×PBS was made for a final concentration of 27% PEG-400, approx. 0.12 mg/mL BSA, 0.01×PBS. This addition led to an increase in voltage by about 200 mV over the course of 200 seconds (slope=0.948) as shown in FIG. 15 (see far right box). The sample then baselined for the remaining 400 seconds (slope=0.154).

### Example 3: Biosensors with Graphene Added Via Contour Ablation

Graphene, a two-dimensional material composed of carbon atoms, has garnered significant attention for its potential application in biosensors due to its unique electrical properties. Despite its promising attributes, however, the non-reproducible nature of graphene biosensors has hindered their widespread commercialization. This issue arises from the difficulty in obtaining a clean graphene layer due to photoresist such as PMMA residues that effect the performance of graphene; etching process which affect the physical and chemical properties of graphene; and variation in the coating of bio-receptors on to graphene. All these issues affect reliable and accurate sensor performance. These challenges have made it difficult for graphene biosensors to reach their full potential as commercial products and have limited their widespread adoption. Thus, a unique contour ablation process has been developed for synthesizing and fabricating graphene based biosensors. The graphene (or other thin material) is transferred onto a pre-patterned substate. During this transfer process, the graphene breaks along the pre-patterned lines and attaches to some areas of the substrate but not others to create the patterned biosensor. This represents a major breakthrough in the commercialization of graphene based biosensors by eliminating several steps in sensor manufacturing and has the added effect of reducing the contamination on graphene which helps preserve sensitivity.

FIGS. 16A-16F and 17A-17F illustrate two variations of the contour ablation process. First, a substrate such as PCB is manufactured various layers, which can include signal electrodes and/or insulating materials, wherein the z-height difference of these layers could range from 50 nanometers to 3 millimeters (FIGS. 16A and 17A). In the second step, the graphene grown on a donor substrate is transferred onto this PCB substrate (FIGS. 16B and 17B). Then, the graphene pattern with defined islands of graphene is shown as the graphene breaks due to the z-height difference on the substrate (FIGS. 16C and 17C). Excess graphene can be washed away, leaving only graphene bound to certain areas of the biosensors, for example, on insulating material laid down on the PCB substate (FIGS. 16D and 17D). Finally, additional insulating materials and electrodes can be printed on top, completing the biosensor (FIGS. 16E and 17E, without a sample, and FIGS. 16F and 17F, with a sample on the biosensor).

To determine if biosensors made with the contour ablation process work, a PCB substrate chip with 24 separate biosensors was made using the contour ablation process. Bioreceptor molecules as described herein were attached on top of graphene and lyophilized with Hemsol™. One application of such sensor was detection of Ricin. The biosensors with patterned graphene were coated with the appropriate insulating and conducting inks and then further functionalized with an aptamer for the ricin toxin. Ricin toxoid was spiked in mud mixed with PBS buffer at various concentrations. Non-specific control spiked in mud were streptococcus endotoxin B (SEB), and bovine serum albumin (BSA). The response of the biosensor is shown in FIG. 17. The lowest detected concentration is 0.28 pM (0.01 ng/ml) with a very low response for off-target SEB and BSA samples.

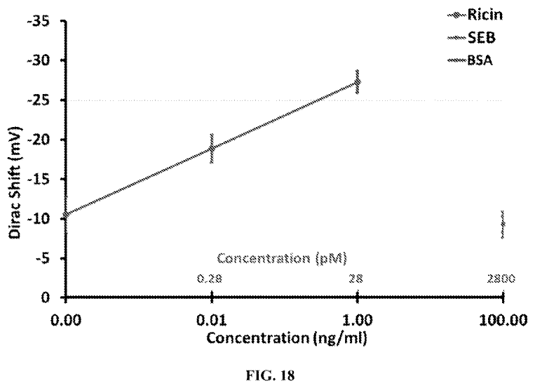

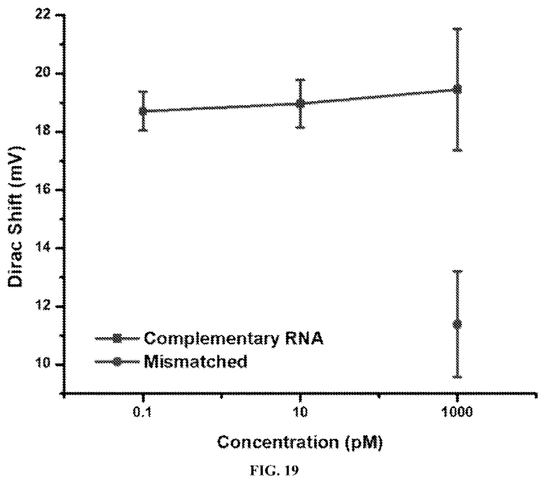

A second application was the detection of porcine reproductive and respiratory syndrome virus (PRRSV). The biosensors were manufactured as described above, except cDNA complementary to viral RNA was functionalized on the biosensors and lyophilized. Viral RNA was spiked in a buffer containing lysis buffer and known negative pig saliva samples. RNA from the influenza virus that is mismatched to the functionalized cDNA was used as a negative control. The response of the biosensor is shown in FIG. 18. There is a drastic difference between complementary and mismatched RNA with complementary RNA having the lowest detectable concentration of 0.1 pM.

Finally, the functionality of the graphene-based biosensor was tested by measuring the distribution of Dirac points. The biosensors were manufactured as described above. The results show that the distribution of Dirac points follows a Gaussian distribution with a median of 80 mV and a very narrow spread of approximately 35 mV. This indicates that most of the Dirac points are tightly clustered around the median value of 80 mV. Furthermore, one standard deviation of the Dirac points is within the range of 50 mV to 120 mV. In contrast, the standard published process of producing graphene-based biosensor shows a much wider distribution of Dirac points, ranging from 250-1500 mV.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A biosensor comprising:
an anti-static substrate comprising a planar surface;
at least one spatially defined active area on the planar surface of the anti-static substrate, each active area comprising a carbon material, a first and a second signal electrode in operable contact with the carbon material, and at least one gate electrode;
a plurality of detecting agents, wherein different detecting agents are positioned at different active areas and immobilized on the deposited carbon material of the active areas; and
an electrical connection comprising a plurality of electrical contacts, each electrical contact configured to transmit an electrical signal between the first and second signal electrodes and at least one gate electrode of a single active area, and the electrical connection.

2. The biosensor of claim 1, wherein:
the carbon material is deposited on the planar surface of the anti-static substrate,
the first and the second signal electrodes are located on opposite sides of the carbon material and are covered by an insulating material, and
the at least one gate electrode is located on top of the insulating material of either the first or the second signal electrodes.

3. The biosensor of claim 1, wherein:
the first and second signal electrodes are deposited on the planar surface of the anti-static substrate, optionally with a bottom insulating material deposited on the planar surface of the anti-static substrate in between the first and the second signal electrodes,
the carbon material is deposited on top of the first and the second signal electrodes, wherein the first and second signal electrodes are located on opposite sides of the carbon material,
a top insulating material is deposited on top of the carbon material on opposite sides of the carbon material and approximately above the first and the second signal electrodes, and
the at least one gate electrode is located on top of the top insulating material above either the first or the second signal electrodes.

4. The biosensor of any one of claims 1-3, wherein the spatially defined array of active areas comprises at least 2 active areas.

5. The biosensor of any one of claims 1-4, wherein the carbon material is graphene, carbon nanotube, or a combination thereof.

6. The biosensor of any one of claims 1-5, wherein the at least two signal electrodes and/or gate electrodes comprise a conductive metal selected from Ti, Cu, Ag, Ir, Pt, Au, or any combination or oxide thereof.

7. The biosensor of any one of claims 1-5, wherein the at least two signal electrodes and/or gate electrodes comprise a carbon-based conducting material selected from carbon nanotubes, graphene oxide, or any combination thereof.

8. The biosensor of any one of claims 1-7, wherein each active area further comprises a preservative solution.

9. The biosensor of any one of claims 1-8, wherein each detecting agent is immobilized on the deposited carbon material via a hydrophobic linker, wherein said hydrophobic linker is coupled to a binding molecule via the detecting agent's amino or carboxy terminus.

10. The biosensor of claim 9, wherein the hydrophobic linker is a peptide linker comprising two or more linker amino acid residues and one or more aromatic amino acid residues

11. The biosensor of claim 10, wherein the two or more linker amino acid residues are selected from glycine, alanine, serine, and combinations thereof.

12. The biosensor of claim 11, wherein the hydrophobic linker comprises a polycyclic aromatic hydrocarbon.

13. The biosensor of any one of claims 1-12, wherein plurality of detecting agents comprise pathogen proteins or peptides thereof, binding molecules capable of binding pathogen proteins of peptides thereof, polynucleotides, or combinations thereof.

14. The biosensor of claim 13, wherein said biosensor further comprises a collection of antibody mimetics, aptamers, DNA molecules, RNA molecules, modified oligonucleotides, or a combination thereof, wherein different members of the collection bind different pathogen proteins and wherein different members of the collection are positioned at different active areas not occupied by the detecting agents, and wherein said members of the collection are immobilized on the deposited carbon material of said active areas.

15. The biosensor of any one of claim 13 or 14, wherein the pathogen is one or more infectious agents selected from a virus, a bacterium, a toxin, or a combination thereof.

16. The biosensor of claim 15, wherein the pathogen is one or more viruses selected from SARS-COV-2, Influenza A, Influenza B, Human papilloma virus, Venezuelan equine encephalitis virus, Vaccinia virus, Ebola virus, Lassa fever virus, Rift Valley fever virus and combinations thereof.

17. The biosensor of claim 15, wherein the pathogen is one or more bacteria selected from the group consisting of Pseudomonas aeruginosa, Neisseria gonorrhoeae, Chlamydia trachomatis, Treponema pallidum, Bacillus anthracis, Yersinia pestis, Francisella tularensis, Burkholderia pseudomallei, Burkholderia mallei, and combinations thereof.

18. The biosensor of claim 15, wherein the pathogen is one or more toxins selected from Ricin toxin, Botulinum Toxin A/B/E, Staphylococcus enterotoxin B (SEB), Abrin toxin, T-2 toxin, B. anthracis LF toxin, B. anthracis EF toxin, B. anthracis PA toxin, and combinations thereof.

19. The biosensor of any one of claims 13-18, wherein each of the plurality of pathogen peptides is between 5 and 50 amino acid residues in length.

20. The biosensor of any one of claims 13-19, wherein the binding molecules of the collection are antibody-based molecules.

21. The biosensor of claim 20, wherein the antibody-based molecules are selected from antibodies, epitope-binding domains thereof, antibody derivatives, antibody mimetics, or combinations thereof.

22. The biosensor of claim 21, wherein the antibody mimetics are selected from the group consisting of an affibodies, affilins, affimers, monobodies, and DARPINs.

23. The biosensor of any one of claims 1-22 further comprising an electromagnet positioned beneath the substrate of the biosensor.

24. The biosensor of any one of claims 1-23, wherein the anti-static substrate comprises a single-layer of anti-static polymeric material.

25. The biosensor of any one of claims 1-24, wherein the anti-static substrate comprises a polymeric material with an anti-static additive.

26. The biosensor of any one of claims 1-25, wherein the anti-static substrate comprises an anti-static printed circuit board.

27. A biosensor system for characterizing a subject's immune response to pathogen exposure, the system comprising:
an electronic reader comprising:
a circuit for delivering a signal; and
a processing device for reading the signal;
a biosensor of any one of claims 1-26 operatively connected to the electronic reader via the electrical connection of the biosensor and configured to receive the signal delivered by the circuit;
wherein the electronic reader is configured to deliver the signal to the biosensor and obtain an output impedance value before and after a sample has been applied to the array of active areas on the biosensor, and said processing device is configured to compare the output impedance values to determine whether a binding event has occurred at one or more of the active areas to characterize the subject's immune response to pathogen exposure.

28. The biosensor system of claim 27, wherein the biosensor comprises an electromagnet positioned beneath the substrate of the biosensor.

29. The biosensor system of claim 27 or 28, further comprising:
a communication interface coupled to the electronic reader for transmitting data from the electronic reader;
a data management computing device configured to receive data from the electronic reader via the communication interface, said data management computing device comprising a memory coupled to a processor which is configured to execute programmed instructions comprising and stored in the memory to geographically map immune response data to pathogen exposure, based on data received from electronic reader.

30. A method of characterizing a subject's immune response to pathogen exposure, said method comprising:
collecting a biological sample from a subject;
providing the biosensor system of any one of claims 27-29;
delivering an electrical signal to the biosensor via the circuit of the electronic reader;
determining a base resistance between the first and second signal electrodes at each active area on the biosensor;
applying the biological sample from the subject to at least one active area on the biosensor, such that the biological sample is in operable contact with the carbon material between the first and second signal electrodes, and the at least one gate electrode at the at least one active area;
identifying a change in the base resistance between the first and second signal electrodes, resulting from applying the biological sample to the at least one active area; and
characterizing the subject's immune response to the pathogen, or the pathogen's antigen profile based on the change in the base resistance between the first and second signal electrodes at the at least one active area.

31. The method of claim 30, wherein the biosensor of the system comprises an electromagnet positioned beneath the substrate of the biosensor, said method further comprising:
labeling, after said collecting, antibodies present in the biological sample with a magnetic moiety;
mixing the biological sample containing the labeled antibodies with a viscous fluid to create a viscous biological sample mixture for said applying;
turning on said electromagnet to localize the labeled antibodies of the biological sample mixture to the active areas on the surface of the substrate during said applying to facilitate binding between labeled antibodies and their cognate detecting agents immobilized on the active surface; and
turning off said electromagnet to release unbound labeled antibodies prior to said identifying.

32. The method of claim 31, wherein said labeling comprises:
contacting the biological sample with an azide containing magnetic moiety, and
exposing the contacted sample with UV light to conjugate the magnetic moiety to antibodies within the biological sample.

33. The method of claim 32, wherein the magnetic moiety is a magnetic bead.

34. The method of claim 33, wherein the magnetic bead is a ferrous oxide magnetic bead.

35. The method of claim 33, wherein the magnetic bead has a diameter of 2 nm to 100 μm.

36. The method of claim 31, wherein the viscous fluid comprises polyethylene glycol (PEG) or glycerin.

37. The method of claim 36, wherein the PEG is PEG-400.

38. The method of claim 36 or 37, wherein the viscous fluid comprises about 20% to about 90% PEG.

39. A biosensor comprising:
an anti-static substrate comprising a planar surface;
at least one spatially defined active area on the planar surface of the anti-static substrate, each active area comprising a carbon material, a first, second, and a third signal electrode in operable contact with the carbon material, and at least one gate electrode;
a plurality of detecting agents, wherein different detecting agents are positioned at different active areas and immobilized on the deposited carbon material of the active areas; and
an electrical connection comprising a plurality of electrical contacts, each electrical contact configured to transmit an electrical signal between the first and second signal electrodes and at least one gate electrode of a single active area, and the electrical connection.

40. The biosensor of claim 39, wherein:
the carbon material is deposited on the planar surface of the anti-static substrate,
the first and the second signal electrodes are located on opposite sides of the carbon material and are covered by an insulating material,
the third signal electrode is located on top of the carbon material, between from the first and the second signal electrodes, and is surrounded by an insulating material, and
the at least one gate electrode is located on top of the insulating material of the first signal electrode.

41. The biosensor of claim 39, wherein:
the first, second, and third signal electrodes are deposited on the planar surface of the anti-static substrate, optionally with a bottom insulating material deposited on the planar surface of the anti-static substrate in between the first, second, and third signal electrodes,
the carbon material is deposited on top of the first, second, and third signal electrodes, wherein the first and second signal electrodes are located on opposite sides of the carbon material, and the third signal electrode is located between from the first and the second signal electrodes,
a top insulating material is deposited on top of the carbon material, approximately above the first, second, and third signal electrodes, and
the at least one gate electrode is located on top of the top insulating material above the first signal electrode.

42. The biosensor of any one of claims 39-41, wherein the spatially defined array of active areas comprises at least 2 active areas.

43. The biosensor of any one of claims 39-42, wherein the carbon material is graphene, carbon nanotube, or a combination thereof.

44. The biosensor of any one of claims 39-43, wherein the at least two signal electrodes and/or gate electrodes comprise a conductive metal selected from Ti, Cu, Ag, Ir, Pt, Au, or any combination or oxide thereof.

45. The biosensor of any one of claims 39-43, wherein the at least two signal electrodes and/or gate electrodes comprise a carbon-based conducting material selected from carbon nanotubes, graphene oxide, or any combination thereof.

46. The biosensor of any one of claims 39-45, wherein each active area further comprises a preservative solution.

47. The biosensor of any one of claims 39-46, wherein each detecting agent is immobilized on the deposited carbon material via a hydrophobic linker, wherein said hydrophobic linker is coupled to a binding molecule via the detecting agent's amino or carboxy terminus.

48. The biosensor of claim 47, wherein the hydrophobic linker is a peptide linker comprising two or more linker amino acid residues and one or more aromatic amino acid residues

49. The biosensor of claim 48, wherein the two or more linker amino acid residues are selected from glycine, alanine, serine, and combinations thereof.

50. The biosensor of claim 49, wherein the hydrophobic linker comprises a polycyclic aromatic hydrocarbon.

51. The biosensor of any one of claims 39-50, wherein plurality of detecting agents comprise pathogen proteins or peptides thereof, binding molecules capable of binding pathogen proteins of peptides thereof, polynucleotides, or combinations thereof.

52. The biosensor of claim 51, wherein said biosensor further comprises a collection of antibody mimetics, aptamers, DNA molecules, RNA molecules, modified oligonucleotides, or a combination thereof, wherein different members of the collection bind different pathogen proteins and wherein different members of the collection are positioned at different active areas not occupied by the detecting agents, and wherein said members of the collection are immobilized on the deposited carbon material of said active areas.

53. The biosensor of any one of claim 51 or 52, wherein the pathogen is one or more infectious agents selected from a virus, a bacterium, a toxin, or a combination thereof.

54. The biosensor of claim 53, wherein the pathogen is one or more viruses selected from SARS-COV-2, Influenza A, Influenza B, Human papilloma virus, Venezuelan equine encephalitis virus, Vaccinia virus, Ebola virus, Lassa fever virus, Rift Valley fever virus and combinations thereof.

55. The biosensor of claim 53, wherein the pathogen is one or more bacteria selected from the group consisting of Pseudomonas aeruginosa, Neisseria gonorrhoeae, Chlamydia trachomatis, Treponema pallidum, Bacillus anthracis, Yersinia pestis, Francisella tularensis, Burkholderia pseudomallei, Burkholderia mallei, and combinations thereof.

56. The biosensor of claim 53, wherein the pathogen is one or more toxins selected from Ricin toxin, Botulinum Toxin A/B/E, Staphylococcus enterotoxin B (SEB), Abrin toxin, T-2 toxin, B. anthracis LF toxin, B. anthracis EF toxin, B. anthracis PA toxin, and combinations thereof.

57. The biosensor of any one of claims 51-56, wherein each of the plurality of pathogen peptides is between 5 and 50 amino acid residues in length.

58. The biosensor of any one of claims 51-57, wherein the binding molecules of the collection are antibody-based molecules.

59. The biosensor of claim 58, wherein the antibody-based molecules are selected from antibodies, epitope-binding domains thereof, antibody derivatives, antibody mimetics, or combinations thereof.

60. The biosensor of claim 59, wherein the antibody mimetics are selected from the group consisting of an affibodies, affilins, affimers, monobodies, and DARPINs.

61. The biosensor of any one of claims 39-60 further comprising an electromagnet positioned beneath the substrate of the biosensor.

62. The biosensor of any one of claims 39-61, wherein the anti-static substrate comprises a single-layer of anti-static polymeric material.

63. The biosensor of any one of claims 39-62, wherein the anti-static substrate comprises a polymeric material with an anti-static additive.

64. The biosensor of any one of claims 39-63, wherein the anti-static substrate comprises an anti-static printed circuit board.

65. A biosensor system for characterizing a subject's immune response to pathogen exposure, the system comprising:
an electronic reader comprising:
a circuit for delivering a signal; and
a processing device for reading the signal;
a biosensor of any one of claims 39-64 operatively connected to the electronic reader via the electrical connection of the biosensor and configured to receive the signal delivered by the circuit;
wherein the electronic reader is configured to deliver the signal to the biosensor and obtain an output impedance value before and after a sample has been applied to the array of active areas on the biosensor, and said processing device is configured to compare the output impedance values to determine whether a binding event has occurred at one or more of the active areas to characterize the subject's immune response to pathogen exposure.

66. The biosensor system of claim 65, wherein the biosensor comprises an electromagnet positioned beneath the substrate of the biosensor.

67. The biosensor system of claim 65 or 66, further comprising:
a communication interface coupled to the electronic reader for transmitting data from the electronic reader;
a data management computing device configured to receive data from the electronic reader via the communication interface, said data management computing device comprising a memory coupled to a processor which is configured to execute programmed instructions comprising and stored in the memory to geographically map immune response data to pathogen exposure, based on data received from electronic reader.

68. A method of characterizing a subject's immune response to pathogen exposure, said method comprising:
collecting a biological sample from a subject;
providing the biosensor system of any one of claims 65-67;
delivering an electrical signal to the biosensor via the circuit of the electronic reader;
applying a control solution to at least one active area on the biosensor, such that the control solution is in operable contact with the carbon material between the first and the third signal electrodes, and the at least one gate electrode at the at least one active area;
determining a base resistance between the second and third signal electrodes at the at least one active area;
applying the biological sample from the subject to the at least one active area, such that the biological sample is in operable contact with the carbon material between the second and third signal electrodes;
identifying a change in the base resistance between the second and third signal electrodes resulting from applying the biological sample to the at least one active area; and
characterizing the subject's immune response to the pathogen, or the pathogen's antigen profile based on the change in the base resistance between the second and third signal electrodes at the at least one active area.

69. The method of claim 68, wherein the biosensor of the system comprises an electromagnet positioned beneath the substrate of the biosensor, said method further comprising:
labeling, after said collecting, antibodies present in the biological sample with a magnetic moiety;
mixing the biological sample containing the labeled antibodies with a viscous fluid to create a viscous biological sample mixture for said applying;
turning on said electromagnet to localize the labeled antibodies of the biological sample mixture to the active areas on the surface of the substrate during said applying to facilitate binding between labeled antibodies and their cognate detecting agents immobilized on the active surface; and
turning off said electromagnet to release unbound labeled antibodies prior to said identifying.

70. The method of claim 69, wherein said labeling comprises:
contacting the biological sample with an azide containing magnetic moiety, and
exposing the contacted sample with UV light to conjugate the magnetic moiety to antibodies within the biological sample.

71. The method of claim 70, wherein the magnetic moiety is a magnetic bead.

72. The method of claim 71, wherein the magnetic bead is a ferrous oxide magnetic bead.

73. The method of claim 71, wherein the magnetic bead has a diameter of 2 nm to 100 um.

74. The method of claim 69, wherein the viscous fluid comprises polyethylene glycol (PEG) or glycerin.

75. The method of claim 74, wherein the PEG is PEG-400.

76. The method of claim 74 or 75, wherein the viscous fluid comprises about 20% to about 90% PEG.

77. A biosensor comprising:
an anti-static substrate comprising a planar surface;
at least one spatially defined active area on the planar surface of the anti-static substrate, each active area comprising a carbon material, a first, a second, a third, and a fourth signal electrode in operable contact with the carbon material, and at least a first and a second gate electrode;
a plurality of detecting agents, wherein different detecting agents are positioned at different active areas and immobilized on the deposited carbon material of the active areas; and
an electrical connection comprising a plurality of electrical contacts, each electrical contact configured to transmit an electrical signal between the first, second, third, and fourth signal electrodes and the at least a first and a second gate electrodes of a single active area, and the electrical connection.

78. The biosensor of claim 77, wherein:
the carbon material is deposited on the planar surface of the anti-static substrate,
the first and the second signal electrodes are located on opposite sides of the carbon material and are covered by an insulating material,
the third signal electrode is located on top of the carbon material, between the first and the fourth signal electrodes, and is surrounded by an insulating material,
the fourth signal electrode is located on top of the carbon material, between the second and the third signal electrodes, and is surrounded by an insulating material,
the at least a first gate electrode is located on top of the insulating material of the first signal electrode, and
the at least a second gate electrode is located on top of the insulating material of the second signal electrode.

79. The biosensor of claim 77, wherein:
the first, second, third, and fourth signal electrodes are deposited on the planar surface of the anti-static substrate, optionally with a bottom insulating material deposited on the planar surface of the anti-static substrate in between the first, second, third, and fourth signal electrodes,
the carbon material is deposited on top of the first, second, third, and fourth signal electrodes, wherein the first and second signal electrodes are located on opposite sides of the carbon material, the third signal electrode is located between the first and the fourth signal electrodes, and the fourth signal electrode is located between the second and the third signal electrodes,
a top insulating material is deposited on top of the carbon material, approximately above the first, second, third, and fourth signal electrodes,
the at least a first gate electrode is located on top of the top insulating material approximately above the first signal electrode, and
the at least a second gate electrode is located on top of the top insulating material approximately above the second signal electrode.

80. The biosensor of any one of claims 77-79, wherein the spatially defined array of active areas comprises at least 2 active areas.

81. The biosensor of any one of claims 77-80, wherein the carbon material is graphene, carbon nanotube, or a combination thereof.

82. The biosensor of any one of claims 77-81, wherein the at least two signal electrodes and/or gate electrodes comprise a conductive metal selected from Ti, Cu, Ag, Ir, Pt, Au, or any combination or oxide thereof.

83. The biosensor of any one of claims 77-81, wherein the at least two signal electrodes and/or gate electrodes comprise a carbon-based conducting material selected from carbon nanotubes, graphene oxide, or any combination thereof.

84. The biosensor of any one of claims 77-83, wherein each active area further comprises a preservative solution.

85. The biosensor of any one of claims 77-84, wherein each detecting agent is immobilized on the deposited carbon material via a hydrophobic linker, wherein said hydrophobic linker is coupled to a binding molecule via the detecting agent's amino or carboxy terminus.

86. The biosensor of claim 85, wherein the hydrophobic linker is a peptide linker comprising two or more linker amino acid residues and one or more aromatic amino acid residues

87. The biosensor of claim 86, wherein the two or more linker amino acid residues are selected from glycine, alanine, serine, and combinations thereof.

88. The biosensor of claim 87, wherein the hydrophobic linker comprises a polycyclic aromatic hydrocarbon.

89. The biosensor of any one of claims 77-88, wherein plurality of detecting agents comprise pathogen proteins or peptides thereof, binding molecules capable of binding pathogen proteins of peptides thereof, polynucleotides, or combinations thereof.

90. The biosensor of claim 89, wherein said biosensor further comprises a collection of antibody mimetics, aptamers, DNA molecules, RNA molecules, modified oligonucleotides, or a combination thereof, wherein different members of the collection bind different pathogen proteins and wherein different members of the collection are positioned at different active areas not occupied by the detecting agents, and wherein said members of the collection are immobilized on the deposited carbon material of said active areas.

91. The biosensor of any one of claim 89 or 90, wherein the pathogen is one or more infectious agents selected from a virus, a bacterium, a toxin, or a combination thereof.

92. The biosensor of claim 91, wherein the pathogen is one or more viruses selected from SARS-COV-2, Influenza A, Influenza B, Human papilloma virus, Venezuelan equine encephalitis virus, Vaccinia virus, Ebola virus, Lassa fever virus, Rift Valley fever virus and combinations thereof.

93. The biosensor of claim 91, wherein the pathogen is one or more bacteria selected from the group consisting of Pseudomonas aeruginosa, Neisseria gonorrhoeae, Chlamydia trachomatis, Treponema pallidum, Bacillus anthracis, Yersinia pestis, Francisella tularensis, Burkholderia pseudomallei, Burkholderia mallei, and combinations thereof.

94. The biosensor of claim 91, wherein the pathogen is one or more toxins selected from Ricin toxin, Botulinum Toxin A/B/E, Staphylococcus enterotoxin B (SEB), Abrin toxin, T-2 toxin, B. anthracis LF toxin, B. anthracis EF toxin, B. anthracis PA toxin, and combinations thereof.

95. The biosensor of any one of claims 90-94, wherein each of the plurality of pathogen peptides is between 5 and 50 amino acid residues in length.

96. The biosensor of any one of claims 90-95, wherein the binding molecules of the collection are antibody-based molecules.

97. The biosensor of claim 96, wherein the antibody-based molecules are selected from antibodies, epitope-binding domains thereof, antibody derivatives, antibody mimetics, or combinations thereof.

98. The biosensor of claim 97, wherein the antibody mimetics are selected from the group consisting of an affibodies, affilins, affimers, monobodies, and DARPINs.

99. The biosensor of any one of claims 77-98 further comprising an electromagnet positioned beneath the substrate of the biosensor.

100. The biosensor of any one of claims 77-99, wherein the anti-static substrate comprises a single-layer of anti-static polymeric material.

101. The biosensor of any one of claims 77-100, wherein the anti-static substrate comprises a polymeric material with an anti-static additive.

102. The biosensor of any one of claims 77-101, wherein the anti-static substrate comprises an anti-static printed circuit board.

103. A biosensor system for characterizing a subject's immune response to pathogen exposure, the system comprising:
an electronic reader comprising:
a circuit for delivering a signal; and
a processing device for reading the signal;
a biosensor of any one of claims 77-102 operatively connected to the electronic reader via the electrical connection of the biosensor and configured to receive the signal delivered by the circuit;
wherein the electronic reader is configured to deliver the signal to the biosensor and obtain an output impedance value before and after a sample has been applied to the array of active areas on the biosensor, and said processing device is configured to compare the output impedance values to determine whether a binding event has occurred at one or more of the active areas to characterize the subject's immune response to pathogen exposure.

104. The biosensor system of claim 103, wherein the biosensor comprises an electromagnet positioned beneath the substrate of the biosensor.

105. The biosensor system of claim 103 or 104, further comprising:
a communication interface coupled to the electronic reader for transmitting data from the electronic reader;
a data management computing device configured to receive data from the electronic reader via the communication interface, said data management computing device comprising a memory coupled to a processor which is configured to execute programmed instructions comprising and stored in the memory to geographically map immune response data to pathogen exposure, based on data received from electronic reader.

106. A method of characterizing a subject's immune response to pathogen exposure, said method comprising:
collecting a biological sample from a subject;
providing the biosensor system of any one of claims 103-105;
delivering an electrical signal to the biosensor via the circuit of the electronic reader;
applying a first control solution to at least one active area on the biosensor, such that the first control solution is in operable contact with the carbon material between the first and the third signal electrodes, and the first gate electrode at the at least one active area;
applying a second control solution to the at least one active area, such that the second control solution is in operable contact with the carbon material between the second and the fourth signal electrodes, and the second gate electrode at the at least one active area;
determining a base resistance between the third and fourth signal electrodes at the at least one active area;
applying the biological sample from the subject to the at least one active area, such that the biological sample is in operable contact with the carbon material between the second and third signal electrodes;
identifying a change in the base resistance between the second and third signal electrodes resulting from applying the biological sample to the at least one active area; and
characterizing the subject's immune response to the pathogen, or the pathogen's antigen profile based on the change in the base resistance between the second and third signal electrodes at the at least one active area.

107. The method of claim 106, wherein the biosensor of the system comprises an electromagnet positioned beneath the substrate of the biosensor, said method further comprising:
labeling, after said collecting, antibodies present in the biological sample with a magnetic moiety;
mixing the biological sample containing the labeled antibodies with a viscous fluid to create a viscous biological sample mixture for said applying;
turning on said electromagnet to localize the labeled antibodies of the biological sample mixture to the active areas on the surface of the substrate during said applying to facilitate binding between labeled antibodies and their cognate detecting agents immobilized on the active surface; and
turning off said electromagnet to release unbound labeled antibodies prior to said identifying.

108. The method of claim 107, wherein said labeling comprises:
contacting the biological sample with an azide containing magnetic moiety, and
exposing the contacted sample with UV light to conjugate the magnetic moiety to antibodies within the biological sample.

109. The method of claim 108, wherein the magnetic moiety is a magnetic bead.

110. The method of claim 109, wherein the magnetic bead is a ferrous oxide magnetic bead.

111. The method of claim 109, wherein the magnetic bead has a diameter of 2 nm to 100 um.

112. The method of claim 107, wherein the viscous fluid comprises polyethylene glycol (PEG) or glycerin.

113. The method of claim 112, wherein the PEG is PEG-400.

114. The method of claim 112 or 113, wherein the viscous fluid comprises about 20% to about 90% PEG.

115. A method of making the biosensor of any one of claims 1-26, 39-64, or 77-102, wherein the method comprising depositing a graphene material on the surface of the biosensor using a contour ablation process.

116. A method of making a biosensor, the method comprising:
obtaining a substrate with at least two layers, wherein there is a z-height difference between the at least two layers,
transferring graphene onto the substrate, wherein the graphene breaks along the at least two layers due to the z-height difference,
washing the biosensor to remove excess graphene,
and, optionally, adding additional layers to the biosensor.

117. The method of claim 116, wherein the z-height difference between the at least two layers is about 50 nanometers to about 3 millimeters.

118. The method of claim 116 or 117, wherein the substrate comprises at least 3 layers.

119. The method of claim 116 or 117, wherein the substrate comprises at least 4 layers.

120. The method of any one of claims 116-119, wherein the additional layers comprise insulating material, or electrode material, or both.
